(19) United States
(12) Patent Application Publication
(10) Pub. No.: US 2026/0166271 A1
(43) Pub. Date: Jun. 18, 2026

(54) CATHETER WITH MULTIPLE INJECTION PORTS

(71) Applicant: Medtronic Ireland Manufacturing Unlimited Company, Dublin (IE)

(72) Inventors: Brian J. Kelly, Oranmore (IE); David J. Hobbins, Galway (IE); Daniel M. King, Galway (IE); Manoj Kumar Singh, Santa Rosa, CA (US); Carlos H. Lima, Santa Rosa, CA (US); Stefan S. Tunev, Santa Rosa, CA (US)

(21) Appl. No.: 19/105,899

(22) PCT Filed: Aug. 23, 2023
(86) PCT No.: PCT/EP2023/073078

§ 371 Date: Feb. 24, 2025

Related U.S. Application Data
(60) Provisional application No. 63/373,385, filed on Aug. 24, 2022.

(51) Int. Cl. A61M 25/00; A61M 25/10

(52) U.S. Cl. CPC A61M 25/0097; A61M 2025/0037; A61M 2025/1004; A61M 2025/105; A61M 2210/12

(57) ABSTRACT

In some examples, a catheter includes a catheter body, an expandable member connected to the catheter body, and a plurality of injection tubes disposed on an outer surface of the expandable member. Each injection tube defines an injection tube lumen and an injection port in fluid communication with the injection tube lumen. The expandable member is configured to expand from a collapsed configuration to an expanded configuration and is configured to not occlude the injection ports of the plurality of injection tubes when the expandable member is in the collapsed configuration.

This application claims the benefit of U.S. Provisional Patent Application Ser. No. 63/373,385, filed 24 Aug. 2022, the entire content of which is incorporated herein by reference.

### TECHNICAL FIELD

The present technology is related to catheters.

### BACKGROUND

Catheters including one or more therapy elements have been proposed for use in various medical procedures, including neuromodulation procedures. For example, some catheters include an energy delivery element, a fluid delivery element, or the like.

### SUMMARY

The present disclosure describes catheters configured to deliver a therapeutic agent to a target treatment site of a patient, as well as systems including the catheters and methods of using the catheters. In some examples, a catheter described herein includes a catheter body which is connected to an expandable member, and a plurality of injection tubes disposed on an outer surface of the expandable member. Each injection tube includes one or more injection ports configured to deliver the therapeutic agent or other substance to tissue of the patient. The expandable member is configured to transform between a collapsed configuration and an expanded configuration. When the expandable member is in the collapsed configuration, the expandable member is configured to not occlude the injection ports of the plurality of injection tubes. For example, the expandable member may not cover the injection ports when the expandable member is in the collapsed, unexpanded state, such that the injection ports remain exposed even when the expandable member is in the collapsed state.

In some examples, the configuration of the expandable member to not occlude the injection ports when the expandable member is in the collapsed configuration may facilitate testing of the catheter, e.g., outside of the patient, while the expandable member is in the collapsed configuration. Because the injection ports are uncovered by the expandable member, fluid can exit the injection ports with minimal to no interaction with the expandable member, even when the expandable member is in a collapsed configuration. In at least this way, the lack of occlusion of the injection ports by the expandable member can reduce any adverse impacts to the expandable member during testing of the plurality of injection tubes and increase the range of tests the clinician may perform on the catheter.

In some examples, a catheter described herein includes an inner member disposed within a catheter body and the expandable member. When the expandable member is in an expanded configuration, a proximal movement of the inner member relative to the expandable member (or the catheter body) causes the expandable member to expand further radially outwards relative to a longitudinal axis of the catheter. The radially outwards movement of the expandable member may place the injection ports of the plurality of the injection tubes in apposition with a wall of a blood vessel of a patient for delivery of therapy to a target treatment site of the patient.

The configuration of the catheters described in this disclosure may improve the efficiency and/or the efficacy therapy delivery to tissue of the patient by at least increasing an area of coverage of the therapy element(s) of the catheter without requiring repositioning of the catheter within the patient. The area of coverage of the therapy element(s) can be represented by, for example, a surface area of wall tissue of a blood vessel affected and/or influenced by the delivered therapeutic agent.

In some examples, the catheters described herein may be useful for neuromodulation within a blood vessel or a body lumen other than a vessel, for extravascular neuromodulation, for non-renal-nerve neuromodulation, and/or for use in therapies other than neuromodulation.

In some examples, the disclosure describes a catheter comprising: a catheter body, an expandable member connected to the catheter body, the expandable member being configured to expand from a collapsed configuration to an expanded configuration; and a plurality of injection tubes disposed on an outer surface of the expandable member, each of the plurality of injection tubes defining an injection tube lumen and an injection port in fluid communication with the respective injection tube lumen, wherein the expandable member is configured to not occlude the injection ports of the plurality of injection tubes when the expandable member is in collapsed configuration.

In another example, the disclosure describes a catheter comprising: a catheter body defining a catheter lumen; an expandable member connected to the catheter body; an inner member disposed within the catheter lumen; and a plurality of injection tubes disposed on an outer surface of the expandable member, each of the plurality of injection tubes defining an injection tube lumen and an injection port in fluid communication with the respective injection tube lumen, wherein in response to proximal movement of the inner member relative to the expandable member while the expandable member is in an expanded configuration, the expandable member is configured to expand radially outwards.

In another example, the disclosure describes a method of delivering therapy to tissue of a patient, the method comprising: navigating a catheter through vasculature of the patient to a target treatment site, wherein the catheter comprises: a catheter body; an expandable member connected to the catheter body; and a plurality of injection tubes disposed on an outer surface of the expandable member, each of the plurality of injection tubes defining an injection tube lumen and an injection port in fluid communication with the respective injection tube lumen; expanding the expandable member of the catheter from a collapsed configuration to an expanded configuration; and delivering therapy to the target treatment site via the injection ports of the plurality of injection tubes, wherein the expandable member does not occlude the injection ports when the expandable member is in the collapsed configuration.

In another example, the disclosure describes a method of delivering therapy to tissue of a patient, the method comprising: navigating a catheter through vasculature of the patient to a target treatment site, wherein the catheter comprise: a catheter body defining a catheter lumen; an expandable member connected to the catheter body; an inner member disposed within the catheter lumen; and a plurality of injection tubes disposed on an outer surface of the expandable member, each of the plurality of injection tubes defining an injection tube lumen and an injection port in fluid communication with the respective injection tube lumen; expanding the expandable member of the catheter from a collapsed configuration to an expanded configuration; retracting the inner member proximally relative to the catheter body, the retraction of the inner member causing the expandable member to expand further radially outwards from the expanded configuration and towards the tissue of the target treatment site; and delivering therapy to the target treatment site via the injection ports of the plurality of injection tubes.

Further disclosed herein is a catheter that includes a catheter body, an expandable member connected to the catheter body, and a plurality of injection tubes disposed on an outer surface of the expandable member, wherein each injection tube defines an injection tube lumen and an injection port in fluid communication with the injection tube lumen, wherein the expandable member is configured to expand from a collapsed configuration to an expanded configuration and is configured to not occlude the injection ports of the plurality of injection tubes when the expandable member is in the collapsed configuration.

The details of one or more examples are set forth in the accompanying drawings and the description below. Other features, objects, and advantages will be apparent from the description and drawings, and from the claims.

### BRIEF DESCRIPTION OF THE DRAWINGS

Reference is made to the attached drawings, wherein elements have the same reference numeral designations represent similar elements throughout.

FIG. 1 is a partial schematic illustration of an example catheter that includes an expandable member and a plurality of injection tubes disposed on an outer surface of the expandable member.

FIG. 2A is a conceptual diagram illustrating a schematic cross-sectional view of an example proximal portion of the catheter of FIG. 1, the cross-section being taken along line A-A in FIG. 1 and in a direction parallel to a longitudinal axis of the catheter.

FIG. 2B is a conceptual diagram illustrating a schematic cross-sectional view of an example distal portion of the catheter of FIG. 1, the cross-section being taken along line A-A in FIG. 1 and in a direction parallel to a longitudinal axis of the catheter.

FIG. 3A is a conceptual diagram illustrating a schematic cross-sectional view of the example distal portion of the catheter of FIG. 2B, the cross-section being taken along line B-B in FIG. 2B and in a direction orthogonal to a longitudinal axis of the catheter.

FIG. 3B is a conceptual diagram illustrating a schematic cross-sectional view of the example distal portion of the catheter of FIG. 2B, the cross-section being taken along line C-C in FIG. 2B and in a direction orthogonal to a longitudinal axis of the catheter.

FIG. 3C is a conceptual diagram illustrating a schematic cross-sectional view of the example distal portion of the catheter of FIG. 2B, the cross-section being taken along line D-D in FIG. 2B and in a direction orthogonal to a longitudinal axis of the catheter.

FIG. 3D is a conceptual diagram illustrating a schematic cross-sectional view of the example expandable member of the catheter of FIG. 2B, the cross-section being taken alone line E-E in FIG. 2B and in a direction orthogonal to a longitudinal axis of the catheter.

FIG. 4A is a conceptual diagram illustrating a schematic cross-sectional view of another example distal portion of the catheter of FIG. 1, the cross-section being taken along line A-A in FIG. 1 and in a direction parallel to a longitudinal axis of the catheter.

FIG. 4B is a conceptual diagram illustrating the example distal portion of the catheter of FIG. 4A, with the expandable member in a radially expanded configuration.

FIG. 5A is a conceptual diagram illustrating a schematic cross-sectional view of another example distal portion of the catheter of FIG. 1, the cross-section being taken along line A-A in FIG. 1 and in a direction parallel to a longitudinal axis of the catheter.

FIG. 5B is a conceptual diagram illustrating a schematic cross-sectional view of the example distal portion of FIG. 5B, the cross-section being taken along line F-F in FIG. 5A and in a direction orthogonal to the longitudinal axis of the catheter.

FIG. 6A is a conceptual diagram illustrating a schematic cross-section view of another example expandable member of the catheter of FIG. 2B, the cross-section being taken along line E-E and in a direction orthogonal to a longitudinal axis of the catheter, with the expandable member in a first collapsed configuration.

FIG. 6B is a conceptual diagram illustrating a view of the expandable member of the catheter of FIG. 6A in a second collapsed configuration.

FIG. 7A is a conceptual diagram illustrating a schematic cross-section view of another example expandable member of the catheter of FIG. 2B, the cross-section being taken along line E-E and in a direction orthogonal to a longitudinal axis of the catheter, with the expandable member in a radially expanded configuration.

FIG. 7B is a conceptual diagram illustrating a view of the expandable member of the catheter of FIG. 7A in a collapsed configuration.

FIG. 8A is a conceptual diagram illustrating a schematic cross-section view of another example expandable member of the catheter of FIG. 2B, the cross-section being taken along line E-E and in a direction orthogonal to a longitudinal axis of the catheter, with the expandable member in a first collapsed configuration.

FIG. 8B is a conceptual diagram illustrating a view of the expandable member of the catheter of FIG. 8A in a second collapsed configuration.

FIG. 9 is a flow diagram illustrating an example method of delivering therapy to a patient using a catheter with multiple injection ports.

FIG. 10 is a conceptual illustration of an example process for accessing a renal artery and modulating renal nerves with the catheter of FIG. 1.

FIG. 11 is a conceptual illustration of an example sympathetic nervous system (SNS) illustrating how the brain communicates with the body via the SNS.

FIG. 12 is an enlarged anatomic view of nerves innervating a left kidney to form the renal plexus surrounding the left renal artery.

FIG. 13 is an anatomic view of a human body depicting neural efferent and afferent communication between the brain and kidneys.

FIG. 14 is a conceptual view of a human body depicting neural efferent and afferent communication between the brain and kidneys.

FIG. 15 is an anatomic view of the arterial vasculature of a human.

FIG. 16 is an anatomic view of the venous vasculature of a human.

### DETAILED DESCRIPTION

In some cases, tissue neuromodulation (e.g., tissue ablation, tissue denervation, or the like) can be accomplished using high pressure chemical ablation. For example, a neuromodulation procedure can involve a high pressure introduction of a therapeutic agent into a wall of a blood vessel for neuromodulation of nearby nerves (e.g., sympathetic renal nerves in the case of a renal denervation procedure). In some cases, the therapeutic agent can be delivered without needles. For example, a therapeutic agent can be delivered at a relatively high pressure through one or more injection ports defined by one or more injection tubes (e.g., a catheter body lumen or a tube separate from a catheter body lumen) positioned in a blood vessel, where the pressure is high enough to force the therapeutic agent into and/or through a blood vessel wall. The therapeutic agent may then diffuse around the target treatment site (e.g., nerves surrounding the blood vessel).

The present disclosure describes catheters configured to deliver a therapeutic agent to a target treatment site within a patient via high pressure delivery of the therapeutic agent to tissue of the patient, which can be needleless. In examples described herein, a catheter includes a therapy delivery element (e.g., one or more injection tubes) at least partially positioned along an outer surface of an expandable member and configured to deliver a therapeutic agent to tissue of a patient. A clinician may navigate the catheter through vasculature of a patient to reach a target treatment site. Once the clinician determines that the catheter is at the target treatment site (e.g., via one or more imaging techniques such as X-ray imaging, fluoroscopy, or the like), the clinician may deliver the therapeutic agent to the target treatment site via one or more injection ports of the catheter.

In some examples, the target treatment site may be an area substantially (e.g., up to 360 degrees) around the wall of the blood vessel (e.g., adjacent blood vessels) of the patient. If a catheter only includes one injection port, then the clinician may need to rotate the catheter within the blood vessel to deliver therapy to the entire target treatment site. The rotation of the catheter may increase a duration of the procedure. The clinician may also misalign the therapy delivery element with the target treatment site which may reduce efficacy of the therapy.

A catheter described herein may be used to deliver a therapeutic agent to tissue at target treatment site without requiring rotation of the catheter within the blood vessel. For example, the catheter can include multiple injection tubes and multiple injection ports positioned to delivery the therapeutic agent to multiple circumferential locations within the blood vessel without requiring rotation and/or repositioning of the catheter.

The expandable member is configured to expand from a collapsed configuration (also referred to herein as a collapsed state) to an expanded configuration (also referred to herein as an expanded state). The collapsed configuration may reduce the profile of the catheter and may facilitate navigation of the catheter within the vasculature of the patient. In the expanded state, the expandable member may place the injection ports in apposition with the vessel wall, which may facilitate penetration of the vessel wall by the therapeutic agent, e.g., without a needle or other physical element that punctures into the vessel wall. In some examples, placing the injection ports in apposition with the vessel wall may reduce an amount of pressure required to deliver the therapeutic agent from the injection ports into the wall tissue (e.g., by reducing an amount of pressure required to cause the therapeutic agent to penetrate the vessel wall).

In some examples, the clinician may wish to test the injection ports positioned on an expandable member of a catheter prior to use of the catheter in the patient and while the expandable member is in a collapsed configuration. If the collapsed expandable member at least partially occludes injection ports of the plurality of injection tubes, then when the clinician expels therapeutic agent from the injection ports (e.g., under a high pressure as may be used during a needleless therapeutic agent delivery process), the expandable member may be adversely impacted, e.g., pierced by the fluid. The possibility of there being adverse impact to the expandable member may reduce the range and/or frequency of tests that a clinician may conduct on the catheter.

The example catheter described in this disclosure may reduce the possibility of adverse impacts to the expandable member from the testing of the injection ports. The example expandable members described in this disclosure may not block (e.g., occlude or otherwise obstruct) the injection ports of the catheter when the expandable member is in a collapsed configuration. When the therapeutic agent is expelled from injection ports, the therapeutic agent may not contact the expandable member and may not adversely impact the expandable member.

The example catheters described in this disclosure are also configured to improve the efficacy of therapy delivery by the catheter compared to catheters with a single injection tube. The example catheters may increase an area of coverage of the catheter without requiring the clinician to rotate or otherwise repositioning the catheter within a blood vessel. The area of coverage may represent a surface area of wall tissue of a blood vessel affected and/or influenced by the delivered therapeutic agent. The example catheters may also be configured to have improved testing capabilities by having an expandable member configured to not occlude the injection ports of the plurality of injection tubes of the example catheters.

Although neuromodulation, and needleless fluid delivery neuromodulation in particular, is primarily referred to herein, the catheters described herein may be used for medical procedures other than neuromodulation, including electrical stimulation therapy. The catheters described herein may also be used in other cavities within a patient.

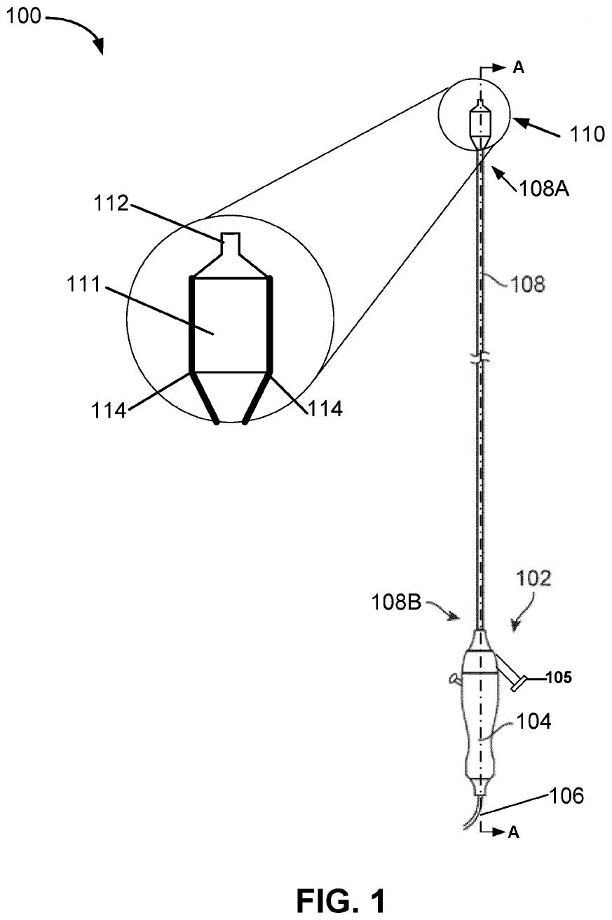

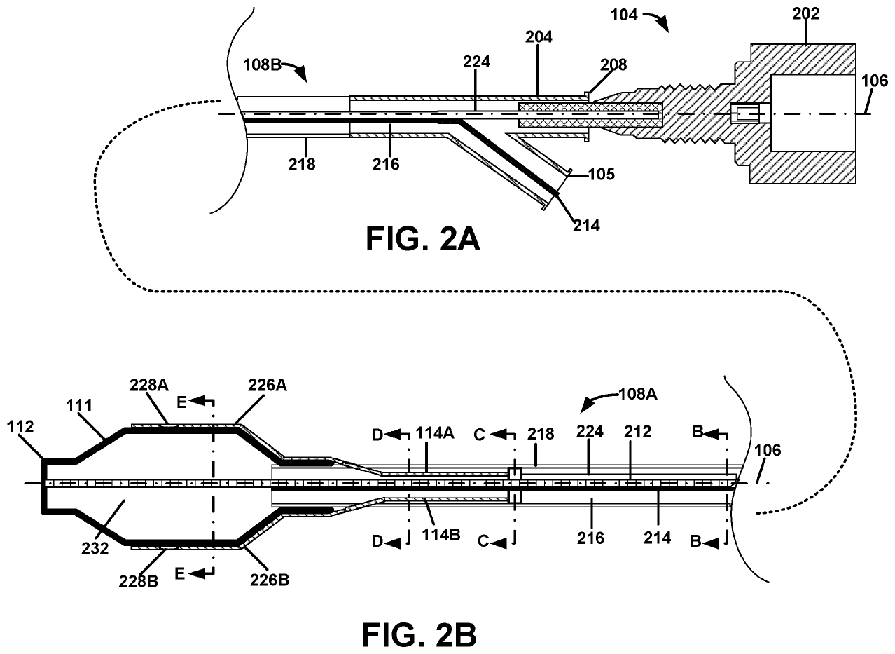

FIG. 1 is a partial schematic illustration of an example catheter 102 that includes a handle 104, an elongated body 108 attached to handle 104, and at least one therapy delivery element 110 carried by elongated body 108. Catheter 102 includes a delivery port 105 configured to receive a fluid (e.g., an ablative chemical or other therapeutic agent) that is delivered via therapy delivery element 110. In some examples, delivery port 105 may be proximal to or defined by handle 104. Therapy delivery element 110 includes expandable member 111 and a plurality of injection tubes 114 (shown in FIGS. 2A and 2B) extending at least partially along an outer surface of expandable member 111. Catheter 102 may be a part of a medical system 100 that includes one or more other components, such as a fluid container that stores a therapeutic agent and a medical device configured to facilitate relatively high pressure delivery of the therapeutic agent from the fluid container to tissue of a patient via catheter 102.

Elongated body 108 includes a distal portion 108A and a proximal portion 108B. Distal portion 108A includes a distal end 112 of catheter 102 and therapy delivery element 110. In some examples, therapy delivery element 110 may be positioned proximal to distal end 112. In other examples, a distal end of therapy delivery element 110 is aligned with distal end 112.

As used herein, the terms “distal” and “proximal” define a position or direction with respect to the treating clinician or clinician's control device (e.g., a handle assembly). “Distal” or “distally” can refer to a position distant from or in a direction away from the clinician or clinician's control device. “Proximal” and “proximally” can refer to a position near or in a direction towards the clinician or clinician's control device. For example, distal portion 108A of elongated body 108 refers to a portion of elongated body 108 at a position distant from the clinician and proximal portion 108B or elongated body 108 refers to a portion of elongated body 108 at a position near the clinician. In some examples, distal portion 108A is a distalmost portion of catheter 102 including a distal end of catheter 102.

Elongated body 108 may have any suitable outer diameter, and the diameter can be constant along the length of elongated body 108 or may vary along the length of elongated body 108. In some examples, elongated body 108 can be 2, 3, 4, 5, 6, or 7 French or another suitable size. An outer layer of elongated body 108 may be formed from any suitable material or combination of materials, such as, but not limited to, one or more of nylon or a thermoplastic such as polyethylene terephthalate (PET), parylene, polyvinyl chloride (PVC), polyethylene, ethylene chlorotrifluoroethylene (ECTFE), or polyvinylidene fluoride (PVDF).

Therapy delivery element 110 includes a plurality of injection tubes 114 positioned along an outer surface of expandable member 111, the plurality of injection tubes 114 being configured to deliver a therapeutic agent at a target tissue site (also referred to herein as “target region” or “treatment site”) within a patient when distal portion 108A is positioned proximate the target tissue site. The therapeutic agent may be a fluid. In some examples, while needleless fluid delivery is primarily referred to herein, in other examples, therapy delivery element 110 can be configured to deliver other types of therapies (e.g., energy such as, but not limited to, ultrasound energy, microwave energy, radiofrequency energy, electrical stimulation, or the like) in addition to a fluid and/or to deliver a fluid via other structures (e.g., fluid delivery using a needle).

Injection tubes 114 are configured to deliver a fluid from proximal portion 108B of catheter 102 (e.g., via delivery port 105) to distal portion 108A and expel a therapeutic agent out of one or more injection ports of injection tubes 114 at a relatively high pressure. The pressure can be selected to enable the therapeutic agent to penetrate the wall of a blood vessel of the patient, e.g., with or without a needle that penetrates into the blood vessel wall. Example pressures include, for example, 5 Megapascal (MPa) to 18 MPa (e.g., about 750 pounds per square inch (psi) to about 2500 psi), such as 10 MPa (or about 10.34 psi). The plurality of injection tubes 114 can be disposed at least partially within elongated body 108 and/or can be positioned entirely external to elongated body 108.

Expandable member 111 is configured to expand radially outwards from a collapsed configuration to an expanded configuration when an inflation fluid, e.g., a saline, is delivered to an interior volume of expandable member 111 via an inflation lumen of catheter 102. Expandable member 111 can be, for example, a balloon (e.g., a compliant balloon). In some examples, when expanded, expandable member 111 is symmetrical (e.g., radially symmetrical and/or longitudinally symmetrical). In some examples, when expanded, expandable member 111 is asymmetrical longitudinally along longitudinal axis 106 and/or radially around longitudinal axis 106.

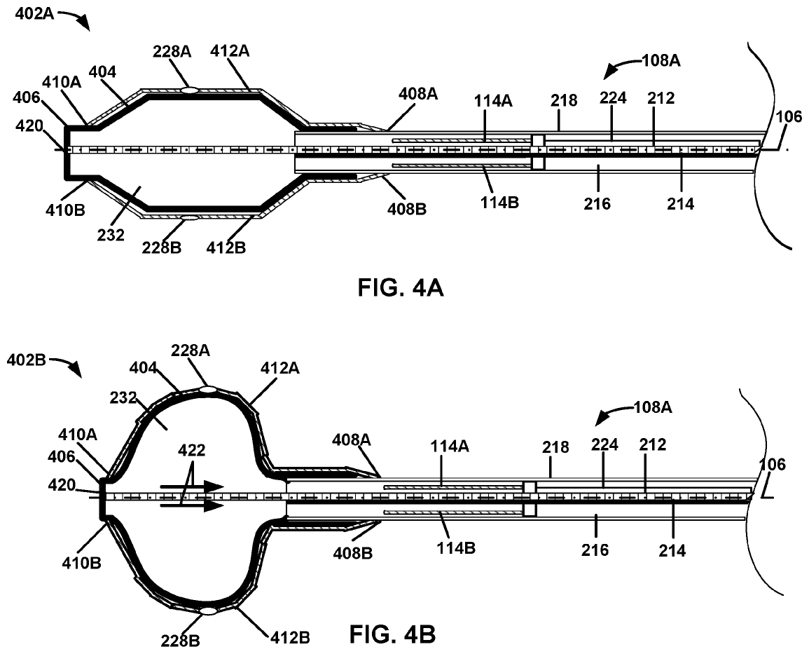

Expandable member 111 is configured such that when expandable member 111 is in the collapsed configuration, expandable member 111 collapses in a manner that leaves injection ports of injection tubes 114 exposed and not occluded. For example, expandable member 111 may form one or more pleats that enables expandable member 111 to fold around longitudinal axis 106 such that the one or more pleats do not occlude or obstruct the injection ports. Example configurations of the one or more pleats in a folded configuration are described in greater detail in FIGS. 5A-7D. By not having the one or more pleats occluding or obstructing the injection ports, injection ports on injection tubes 114 may be tested while expandable member 111 is in the collapsed configuration without adversely impacting (e.g., piercing) expandable member 111.

As discussed in further detail with reference to FIGS. 2A and 2B, elongated body 108 includes an outer catheter body which defines an outer catheter lumen. In some examples, the outer catheter lumen is an inflation lumen in fluid communication with an inner volume of expandable member 111. A clinician may introduce an inflation fluid into and/or extract the inflation fluid from the inner volume of expandable member 111 via the outer catheter lumen, e.g., via a port on proximal portion 108B. In some examples, the port may be proximal to or defined by handle 104. In other examples, an inflation tube separate from the outer catheter body (e.g., extending through the outer catheter lumen) can be used to deliver an inflation fluid to the inner volume of expandable member 111 to expand expandable member 111 to an expanded state.

Introduction of the inflation fluid into the inner volume of expandable member 111 expands expandable member 111 from a relatively low profile collapsed configuration to an expanded configuration. The inflation fluid (also referred to as the “fluid”) is biocompatible. In some examples, the fluid may be a liquid such as sterile water or saline. In addition, in some examples, the fluid includes a contrast media, e.g., mixed with sterile water. The fluid can be removed from the inner volume of expandable member 111 to transition expandable member 111 from the expanded configuration into the collapsed configuration. The clinician may transform expandable member 111 between the expanded configuration and the collapsed configuration, e.g., to navigate catheter 102 between multiple target regions and to deliver therapeutic agents at the multiple target regions.

In some examples, catheter 102 is configured to accommodate a variety of vessel diameters. For example, renal vessels may have a diameter between about 3 mm and about 8 mm. Other vessels may have other diameters. Distal portion 108A may accommodate different vessel diameters by having expandable member 111 expand to different diameters. In this way, a single catheter 102 may be used to deliver therapy to vessels with different diameters, e.g., diameters in a range of between about 2 mm and about 10 mm, such as in a range of about 3 mm to about 8 mm.

Distal portion 108A of elongated body 108 is configured to be advanced within a hollow anatomical structure (e.g., a blood vessel) of a human patient to locate expandable member 111 at a target region (e.g., a target treatment site) within or otherwise proximate to the hollow anatomical structure. For example, elongated body 108 may be configured to position the plurality of injection tubes 114 within a blood vessel, a ureter, a urethra, a duct, an airway, or another naturally occurring lumen within the human body. The examples described herein focus on the anatomical structure being a blood vessel, such as a renal vessel, but it will be understood that similar techniques may be used with other hollow anatomical structures.

In certain examples, intravascular delivery of catheter 102 includes percutaneously inserting a guidewire (not shown in FIG. 1) into a vessel of a patient and moving elongated body 108 along the guidewire or another guide member until expandable member 111 reaches a target treatment site (e.g., a renal artery). For example, catheter 102 can include a guidewire tube or another structure that defines a guidewire lumen configured to receive a guidewire for delivery of catheter 102 using over-the-wire (OTW) or rapid exchange (RX) techniques. In still other examples, catheter 102 can be configured for delivery via an inner catheter or sheath (not shown in FIG. 1), or other inner member (e.g., a guide catheter). In addition to or instead of an inner guide member, in some examples, catheter 102 is a steerable (e.g., with one or more pull wires connected to distal portion 108A and manipulatable by the clinician) or non-steerable catheter configured for use without a guidewire. Thus, in some examples, the inner member may be a navigation wire (e.g., a guidewire or the like), which can be disposed on an outer surface of elongated body 108 of catheter 102 or extend within an inner lumen of catheter 102.

Once at the target treatment site, the clinician may transition expandable member 111 from the collapsed configuration into the expanded configuration by introducing the fluid into the inner volume of expandable member 111, e.g., via the outer catheter lumen or via an inner tube disposed within the outer catheter lumen. Once expandable member 111 is in the expanded configuration, the clinician may operate a medical device to deliver therapeutic agent to the target treatment site via injection tubes 114 disposed on expandable member 111. The therapeutic agent can, for example, provide or facilitate neuromodulation therapy at the target treatment site. For ease of description, the following discussion will be primarily focused on delivering therapeutic agents. It will be understood, however, that catheter 102 may include elements configured to deliver other types of therapies (e.g., radiofrequency (RF) ablation, microwave ablation, ultrasound ablation, cryoablation, and the like).

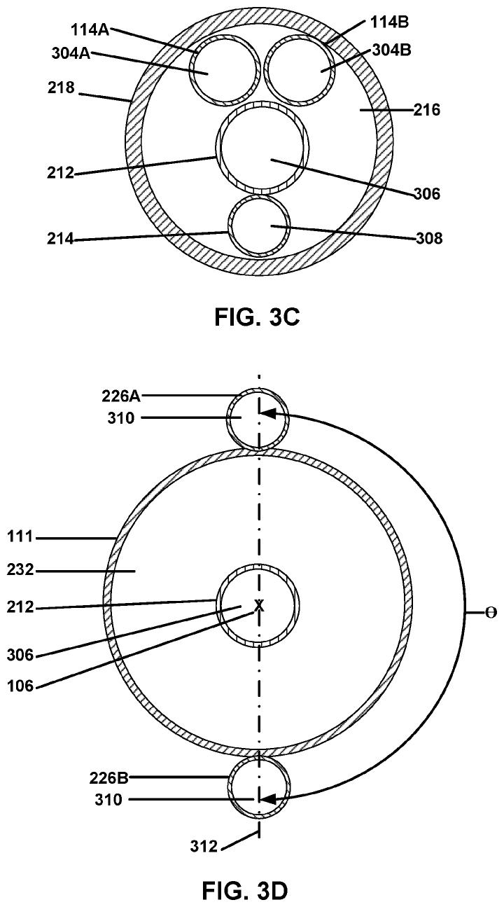

In some examples, as discussed with reference to FIGS. 4A and 4B, after the clinician transitions expandable member 111 into the expanded configuration, the clinician may retract an inner member (e.g., the guide member) proximal to elongated body 108 of catheter 102. The retraction of the inner member may further cause distal end of expandable member 111 to deflect and cause portions of expandable member 111 to expand radially outwards of catheter 102. In some examples, the expansion of expandable member 111 expands injection tubes 114 radially outwards of catheter 102 (e.g., by expandable member 111 pushing injection tubes 114 away from longitudinal axis 106) and causes injection tubes 114 to be in apposition with the wall of the blood vessel. In some examples, the expansion of expandable member 111 places injection ports disposed on injection tubes 114 in apposition with the vessel wall. Placing injection ports in apposition with the vessel wall may reduce pressure required to deliver therapeutic agent to the wall tissue due to improved contact between the injection ports and the vessel wall.

FIG. 2A is a conceptual diagram illustrating a cross-sectional view of an example proximal portion 108B of the catheter 102 of FIG. 1, the cross-section being taken along line A-A in FIG. 1 and in a direction parallel to a longitudinal axis of catheter 102. As illustrated in FIG. 2A, in some examples, handle 104 includes connector 202 and housing 204 including delivery port 105. Housing 204 is directly or indirectly mechanically connected to proximal portion 108B of elongated body 108. Thus, a clinician can manipulate elongated body 108 by at least manipulating housing 204 (e.g., distally pushing housing 204 relative to a patient, proximally withdrawing housing 204 relative to the patient, rotating housing 204 about a longitudinal axis of elongated body 108, and the like). Elongated body 108 defines a catheter lumen 216 in which fluid delivery tube 224 is positioned. Inner tube 214 is also positioned in catheter lumen 216.

Connector 202 may be attached to a proximal end 208 of housing 204 (e.g., a hub) and may be in fluid communication with fluid delivery tube 224. Fluid delivery tube 224 is in fluid communication with injection tubes 114 (e.g., as illustrated in FIG. 2B) and defines a fluid pathway through which a therapeutic agent can flow from connector 202 to injection tubes 114. Injection tubes 114 are configured to deliver the therapeutic agent to tissue of the patient via one or more injection ports defined by injection tubes 114 (e.g., as illustrated in FIG. 2B). Each of injection tubes 114 may define one or more injection ports and, in some examples, the injection ports of some or all of injection tubes 114 can be located at a distal portion of the respective injection tubes 114.

Connector 202 is configured to fluidically connect fluid delivery tube 224 to a pressure source (e.g., a compressor, a pump, a gas canister, or the like) and a therapeutic agent source of system 100 to facilitate delivery of the therapeutic agent to tissue of a patient via the one or more injection ports of injection tubes 114 connected to fluid delivery tube 224. Connector 202 may be permanently and/or removable fluidically connected to a proximal portion of fluid delivery tube 224. In some examples, connector 202 is attached to housing 204 or can be separate from housing 204.

Housing 204 includes delivery port 105, which is in fluid communication with an inner volume 232 (FIG. 2B) of expandable member 111. A clinician can introduce and/or extract inflation fluid from within inner volume 232 of expandable member 111 via delivery port 105. For examples, delivery port 105 can be in fluid communication with catheter lumen 216 (either directly or indirectly), which defines a fluid pathway between delivery port 105 and inner volume 232 of expandable member 111. As another example, catheter 102 may further include an inner tube 214 that is in fluid communication with inner volume 232 of expandable member 111. Inner tube 214 may enter housing 204 and catheter lumen 216 through delivery port 105 and the clinician may access inner tube 214 through delivery port 105.

Handle 104 may additionally include one or more components not pictured in FIG. 2A including, but are not limited to, structures to facilitate delivery of a therapeutic agent to tissue via therapy delivery element 110. For example, handle 104 can include a trigger to initiate delivery of a therapeutic agent into injection tubes 114 and/or fluid delivery tube 224, a visualization window, a safety collar, a cam clamp, or the like. The clinician may operate the one or more components to deliver the therapeutic agent to inspect the therapeutic agent within handle 104, to test injection ports of injection tubes 114, to prevent unintended delivery of the therapeutic agent, or the like. In some examples, elongated body 108 may be secured to housing 204, e.g., via a cam clamp disposed on housing 204.

FIG. 2B is a conceptual diagram illustrating a schematic cross-sectional view of an example distal portion 108A of the catheter 102 of FIG. 1, the cross-section being taken along line A-A in FIG. 1 and in a direction parallel to a longitudinal axis of catheter 102. Elongated body 108 may be defined by outer catheter body 218, which defines catheter lumen 216. In some examples, distal portion 108A of elongated body 108 is connected to a proximal end of expandable member 111. In some examples, as illustrated in FIG. 2B, elongated body 108 includes a plurality of injection tubes 114A, 114B (collectively referred to as “injection tubes 114”) at least partially disposed within catheter lumen 216 of elongated body 108. Guidewire tube 212, inner tube 214, at least a portion of injection tubes 114, and at least a portion of fluid delivery tube 224 may be disposed within a distal portion of catheter lumen 216.

Guidewire tube 212 may extend through expandable member 111 and may be connected to distal end 112 of catheter 102. Guidewire tube 212 defines a guidewire lumen configured to receive one or more inner members (e.g., a guidewire, a guiding member, or the like). A clinician may navigate catheter 102 through vasculature of the patient with the aid of the inner member received in guidewire tube 212. In some examples, a proximal opening to guidewire tube 212 is a handle 104 and/or a point proximal to handle 104. In some examples, a proximal opening to guidewire tube 212 is at a point distal to handle 104 (e.g., via a RX port). Guidewire tube 212 may include a relatively flexible material (e.g., a relatively soft polymer such as high-density polyethylene (HDPE)) and can include a lubricious inner lumen.

Injection tubes 114 are disposed within catheter lumen 216 and are fluidically connected to fluid delivery tube 224. As illustrated in FIG. 2B, in some examples, fluid delivery tube 224 terminates and separates into a plurality of injection tubes 114 at a first point proximal to the proximal end of expandable member 111. In other examples, a plurality of injection tubes 114 is separate from fluid delivery tube 224 and mechanically and fluidically coupled to a distal portion of fluid delivery tube 224. For example, a proximal portion of injection tubes 114 can be positioned within an inner lumen defined by fluid delivery tube 224, such that fluid can flow from the inner lumen defined by fluid delivery tube 224 into inner lumens of injection tubes 114. The distal end of fluid delivery tube 224 can be sealed such that the fluid does not flow past the distal end of fluid delivery tube 224 except through lumens of injection tubes 114.

Injection tubes 114 exit catheter lumen 216 at a second point proximal to expandable member 111 and external portions 226A, 226B (collectively referred to as “external portions 226”) of injection tubes 114A, 114B, respectively, are disposed on an outer surface of expandable member 111. In some examples, as illustrated in FIG. 2B, catheter 102 includes two injection tubes 114. In some examples, catheter 102 may include three or more injection tubes 114, such as three, four, five, six or more injection tubes. In some examples, injection tubes 114 are equally spaced around an outer perimeter of expandable member 111. In other examples, injection tubes are unequally spaced around the outer perimeter. For example, injection tubes 228 may be closer together around a first portion of expandable member 111 and farther apart around a second portion of expandable member 111.

External portions 226 each define one or more injection ports. In the example shown in FIG. 2B, external portions 226 define respective injection ports 228A, 228B (collectively referred to as “injection ports 228”). As illustrated in FIG. 2B, in some examples, each of external portions 226 (e.g., external portion 226A) may define only one of injection ports 228 (e.g., injection port 228A). In other examples, each of external portions 226 may define two or more of injection ports 228 arranged longitudinally along the respective external portion of external portions 226. When expandable member 111 is in the expanded configuration, and in a blood vessel of a patient, expandable member 111 positions external portions 226 and/or injection ports 228 in apposition with a wall of the blood vessel. When expandable member 111 transitions to the collapsed configuration, external portions 226 may remain connected to the outer surface of expandable member 111 and may contract radially inwards away from the blood vessel wall alongside expandable member 111. This enables catheter 102 to assume a relatively low profile configuration for navigation through vasculature of the patient. In some examples, external portion 226 may include one or more radiopaque markers to facilitate visualization of injection ports 228 and aid in delivery of the therapy to tissue of the patient.

In some examples, when expandable member 111 is in the collapsed configuration, expandable member 111 does not occlude injection ports 228 on injection tubes 114. For example, expandable member 111 may form one or more pleats and fold around longitudinal axis 106 such that the one or more pleats do not occlude or obstruct injection ports 228, e.g., as illustrated in FIGS. 5A-7D. Injection ports 228 on injection tubes 114 may be tested while expandable member 111 is in the collapsed configuration without adversely impacting expandable member 111 due to expandable member 111 not occluding or obstructing injection ports 228.

For example, expandable member 111 may define one or more pleats, between which external portions 226 may be located. When expandable member 111 is in the collapsed configuration, the one or more pleats are configured to fold towards longitudinal axis 106 of catheter 102 and do not block (e.g., occlude, cover, overlap with in a radial direction, or otherwise obstruct) injection ports 228. A clinician may eject therapeutic agent from injection ports 228 while expandable member 111 is in the collapsed configuration, e.g., to test functionality of injection ports 228, external portion 226, injection tubes 114, and/or fluid delivery tube 224. Due to the manner in which the pleats of expandable member 111 are folded to not obscure injection ports 228, the therapeutic agent will not interact with or adversely affect the one or more pleats of expandable member 111 while the therapeutic agent is being delivered from injection ports 228 while expandable member 111 is in the collapsed configuration.

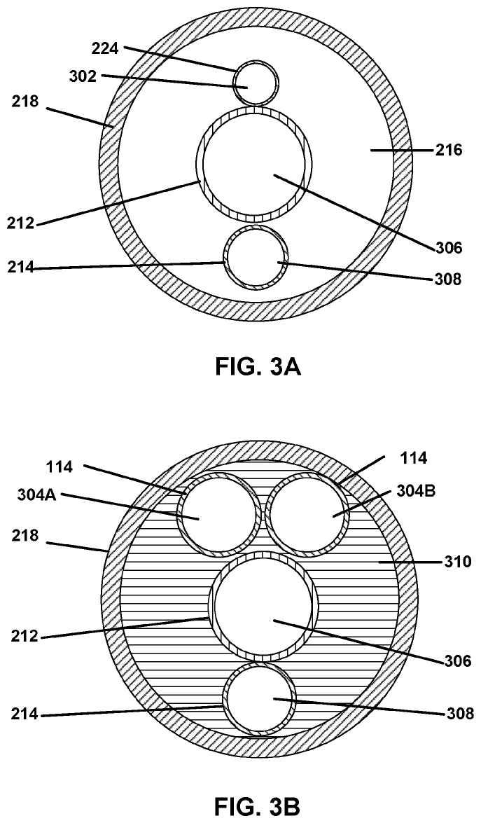

FIGS. 3A-3C illustrate conceptual and schematic cross-sectional views of distal portion 108A of catheter 102 of FIG. 2B at different points along distal portion 108A of elongated body 108. In particular, the cross-sections are taken along line B-B, C-C, and D-D in FIG. 2B, respectively, in a direction orthogonal to longitudinal axis 106.

As illustrated in FIGS. 3A-3C, outer catheter body 218 of elongated body 108 defines catheter lumen 216. Outer catheter body 218 may have any suitable thickness, defined in a radial direction between the outer surface of outer catheter body 218 and the inner surface of outer catheter body 218 defining outer catheter lumen 216. In some examples outer catheter body 218 has a thickness of 0.05 millimeters (mm) to about 0.3 mm (e.g., about 0.002 inches to about 0.010 inches).

At line B-B, as shown in FIG. 3A, fluid delivery tube 224, guidewire tube 212, and inner tube 214 are disposed within catheter lumen 216. Fluid delivery tube 224 defines fluid delivery tube lumen 302 and is in fluid communication with connector 202 and injection tubes 114. Fluid delivery tube lumen 302 may be in fluid communications with multiple injection tubes 114, multiple external portions 226, and multiple injection ports 228 disposed on expandable member 111. Fluid delivery tube 224 can have any suitable configuration that facilitates relatively high pressure fluid injection, as well as to facilitate deliver of catheter 102 through vasculature of a patient to a target region. In some examples, fluid delivery tube 224 may withstand an internal pressure at least 18 MPa and may be resistant to frequent changes in pressure.

Guidewire tube 212 defines guidewire lumen 306. As discussed above, a clinician may access guidewire lumen 306, e.g., via a rapid exchange (RX) port disposed along elongated body 10 or at a point closer to or at a proximal end of catheter 102. Inner tube 214 defines inner tube lumen 308, which, as discussed above, can be used to deliver an inflation fluid into and remove the inflation fluid from inner volume 232 of expandable member 111. In some examples, however, catheter 102 does not include inner tube 214 and the inflation fluid is delivered to inner volume 232 of expandable member 111 via catheter lumen 216.

At a point along line C-C in FIG. 2B, e.g., as illustrated in FIG. 3B, fluid delivery tube 224 separates into injection tubes 114. In some examples, a plurality of fluid delivery tubes 224 may be connected to injection tubes 114. Injection tubes 114 may define injection tube lumens 304 A-B (collectively referred to as “injection tube lumens 304”). Injection tube lumens 304 are in fluid connection with fluid delivery tube lumen 302 and are configured to receive a therapeutic agent at relatively high pressures. Injection tubes 114 have any suitable configuration that facilitates relatively high pressure fluid injection, but are relatively flexible to facilitate navigation of catheter 102 through vasculature of a patient to a target treatment site. In some examples, injection tubes 114 are configured to withstand at least 18 MPa and may be resistant to frequent changes in pressure.

As shown in FIG. 3B, catheter 102 may include separation member 310 obstructing a void in catheter lumen 216 between guidewire tube 212, inner tube 214, and injection tubes 114. Separation member 310 may restrict flow of fluids (e.g., blood) through catheter lumen 216 and outside one of guidewire tube 212, inner tube 214, and/or injection tubes 114. Separation member 310 may facilitate flow of therapeutic agents between fluid delivery tube lumen 302 and injection tube lumens 304. In some examples, separation member 310 prevents flow of therapeutic agents out of fluid delivery tube lumen 302 and into catheter lumen 216.

Along line D-D in FIG. 2B, as illustrated in FIG. 3C, separate injection tubes 114 are disposed within catheter lumen 216 along with guidewire tube 212, inner tube 214, e.g., in accordance with the descriptions above. In some examples, as illustrated in FIGS. 3B and 3C, injection tubes 114 may be positioned relatively close together within catheter lumen 216. In other examples, injection tubes 114 may be positioned within catheter lumen 216 and up to 180 degrees apart.

FIG. 3D is a conceptual diagram illustrating a cross-sectional view of the example expandable member 111 of the catheter 102 of FIG. 2B, the cross-section being taken alone line E-E in FIG. 2B. As illustrated in FIG. 3D, external portions 226 each defines a respective external injection tube lumens 310A, 310B (collectively referred to “external injection tube lumens 310”). External portion 226 are disposed radially around an outer surface of expandable member 111. Adjacent external portions 226 may separated around expandable member 111 by angle ⊖. Angle ⊖ may be between about 90 degrees to about 180 degrees in either the clockwise or the counter-clockwise direction. Change a value of angle ⊖ may cause adjustments of areas of influence of each of external portions 226, e.g., by changing the orientation of injection ports 228 within a blood vessel. For example, a smaller value of angle ⊖ may cause the areas of influence of adjacent external portions 226 to become closer together and/or overlap, and vice versa. In some examples, injection ports 228 are oriented away from anatomical structures within the patient (e.g., away from a renal vein while catheter 102 is disposed within a renal artery).

In some examples in which angle ⊖ is 180 degrees, each of injection ports 228 may have an area of influence of 180 degrees around the vessel perimeter and injection ports 228 in conjunction deliver the therapeutic agent 360 degrees around the vessel perimeter. For example, once delivered to tissue around the blood vessel through the vessel wall from multiple injection ports located at different circumferential locations of the inner perimeter of the blood vessel, the therapeutic (e.g., ablative) agent may diffuse through tissue around the blood vessel. In other examples, injection ports 228 in conjunction deliver the therapeutic agent less than 360 degrees (e.g., 270 degrees or less) around the vessel perimeter. A single injection port 228 may, for example, deliver the therapeutic agent about 270 degrees (e.g., within about 0-15 degrees of 270 degrees) around the vessel perimeter due to the spread of the injectate within the tissue once released from the respective injection port 228.

Multiple injection tubes 114 each defining at least one injection port can also enable the use of less pressure to penetrate into or through the wall of a blood vessel, compared to delivery of a therapeutic agent via a single injection tube with a single injection port.

FIG. 4A is a conceptual diagram illustrating a cross-sectional view of another example distal portion 108A of the catheter 102 of FIG. 1, the cross-section being taken along line A-A in FIG. 1 in a direction parallel to longitudinal axis 206 of catheter 102. Expandable member 404 shown in FIG. 4A is an example of expandable member 111 of FIG. 1. FIG. 4B is a conceptual diagram illustrating the example distal portion 108A of the catheter 102 of FIG. 4A, with expandable member 404 in a radially expanded configuration. FIG. 4A illustrates distal portion 108A of catheter 102 with expandable member 404 in a first expanded configuration 402A and FIG. 4B illustrates expandable member 404 in a second expanded configuration 402B. Expandable member 404 may be configured to not block (e.g., not occlude or otherwise obstruct) injection ports 228 when expandable member 404 in some, but not all, examples.

Expandable member 404 has a distal end 406 and defines inner volume 232 configured to receive an inflation fluid to expand expandable member 404 from a collapsed configuration to the first expanded configuration 402A. Injection tubes 114 are disposed on an outer surface of expandable member 404 and extend longitudinally along expandable member 404. Expandable member 404 may be connected to elongated body 108 (e.g., to outer catheter body 218). Elongated body 108 may contain guidewire tube 212, inner tube 214, injection tubes 114, and/or fluid delivery tube 224, e.g., in accordance with example catheters described in FIGS. 2A-3C.

In the example shown in FIGS. 4A and 4B, injection tubes 114 exit outer catheter body 218 via ports 408A-B (collectively referred to as “ports 408”) defined by elongated body 108, ports 408 being located proximal to expandable member 404. In some examples, ports 408 may be located at a distal end of elongated body 108. Each of injection tubes 114 includes a respective external portion 412A, 412B (collectively referred to as “external portions 412”) extending from ports 408 and affixed to distal end 406 of expandable member 404 at respective fixation points 410A, 410B (collectively referred to as “fixation points 410”). In some examples, as illustrated in FIG. 4A, there may be a gap between the outer surface of expandable member 404 and external portions 412. In other examples, external portions 412 may be in contact with the outer surface of expandable member 404.

Guidewire tube 212 may be relatively rigid, e.g., compared to injection tubes 114, and is configured to advance distally or retract proximally relative to elongated body 108 in response to an exertion of force on guidewire tube 212, e.g., by the clinician via handle 104 and/or via a proximal end of guidewire tube 212. Guidewire tube 212 is mechanically connected secured to a distal portion of expandable member 404, such as to distal end 406 of expandable member 404 at fixation point 420. This mechanical connection enables a proximal force 422 exerted on guidewire tube 212 to cause guidewire tube 212 to exert proximal force 422 on distal end 406 of expandable member 404 and cause distal end 406 of expandable member 404 to retract proximally relative to elongated body 108 of catheter 102, as shown in FIG. 4B.

As distal end 406 of expandable member 404 retracts proximally relative to elongated body 108, the retraction causes expandable member 404 to expand further radially outwards from first expanded configuration 402A to second expanded configuration 402B, e.g., towards a blood vessel wall. External portions 412 of injection tubes 114 are secured to expandable member 404, e.g., to distal end 406 at fixation points 410, such that the radially outward expansion of expandable member 404 causes external portions 412 to further expand radially outwards, e.g., as illustrated in FIG. 4B. The further expansion of external portions 412 may place injection ports 228A in apposition with the wall tissue of the blood vessel.

In some examples, external portions 412 of injection tubes 114 include materials configured to flexibly expand radially outwards. Example materials and structures include, but are not limited to, braided polymer tubes, which may be configured to form the further-radially-expanded configuration and withstand pressure within injection tubes 114. In some examples, external portions 412 include polyimide and a braided polymer tube including one or more of Kevlar, polyethylene naphthalate (PEN), braided polymer wires (e.g., nylon braided wires), or the like. A combined strength of the braided polymer tubes and a polymer outer jacket can provide sufficient strength for the external portions 412 to withstand the pressures within injection tubes 114 while forming the further-radially-expanded configuration. In some examples, external portions 412 include a polytetrafluoroethylene (PTFE) inner liner disposed radially inwards of the braided polymer tubes.

In some examples, expandable member 404 and external portions 412 are configured to expand radially outwards in a symmetrical manner. In some examples, expandable member 404 and external portions 412 are configured to expand radially outwards in an asymmetrical manner (e.g., expandable member 404 and external portions 412 expand radially outwards in a first direction but not in a second direction). In some examples, in the radially expanded configuration 402B, expandable member 404 is radially and/or longitudinally symmetrical. In some examples, in configuration 402B, expandable member 404 is radially and/or longitudinally asymmetrical.

FIG. 5A is a conceptual diagram illustrating a schematic cross-sectional view of another example distal portion 422 of catheter 102 of FIG. 1, the cross-section being taken along line A-A in FIG. 1 and in a direction parallel to longitudinal axis 106 of catheter 102. FIG. 5B is a conceptual diagram illustrating a schematic cross-sectional view of distal portion 422 of catheter 102 of FIG. 5A, the cross-section being taken along line F-F in FIG. 5A and in a direction orthogonal to longitudinal axis 106 of catheter 102. In contrast to the example of FIGS. 4A and 4B, injection tubes 114 exit elongated body 108 via distal end 424 of elongated body 108, rather than from side ports 408A, 408B.

As illustrated in FIG. 5A, expandable member 111 may be distal to elongated body 108. External portions 226 of injection tubes 114 extend from distal end 424 of elongated body 108 and extend along the outer surface of expandable member 111. In some examples, expandable member 111 is configured to be advanced and/or retracted relative to elongated body 108 via distal and/or proximal movement of guidewire tube 212 along longitudinal axis 106, respectively. In other examples, expandable member 111 has a fixed longitudinal position relative to elongated body 108.

In the example shown in FIG. 5A, expandable member 111 includes a bond tube 430 secured to a proximal portion (including proximal end 428) of expandable member 111. Bond tube 430 may define a profile of proximal end 428 of expandable member 111. In some examples, bond tube 430 has an outer diameter that is less than the inner diameter of catheter lumen 216 and may be at least partially disposed within catheter lumen 216 of elongated body 108. Proximal end 426 of bond tube 430 may be sealed around guidewire tube 212 and inner tube 214, e.g., to isolate inner volume 232 from the vasculature of the patient.

As illustrated in FIG. 5B, external portions 226 of injection tubes 114 are disposed on the outer surface of expandable member 111. External portions 226 may define an outer profile of catheter 102 proximal to expandable member 111. In some examples, as shown in FIG. 5B, external portions 226, bond tube 430, and/or a proximal portion of expandable member 111 may have an oval cross-sectional shape to reduce the outer profile of catheter 102 while expandable member 111 is in the collapsed configuration. The example configuration of external portions 226 illustrated in FIG. 5 may correspond to examples in which external portions 412 include polyimide and a braided polymer shaft including braided polymer wires, as described in greater detail above in FIG. 4B.

Bond tube 430 defines a part of catheter lumen 432. Catheter lumen 432 may retain guidewire tube 212 and inner tube 214 and may be in fluid communication with inner volume 232 of expandable member 111. In some examples, catheter lumen 432 may be closed at proximal end 426, e.g., to isolate catheter lumen 432 and inner volume 232 from the vasculature of the patient.

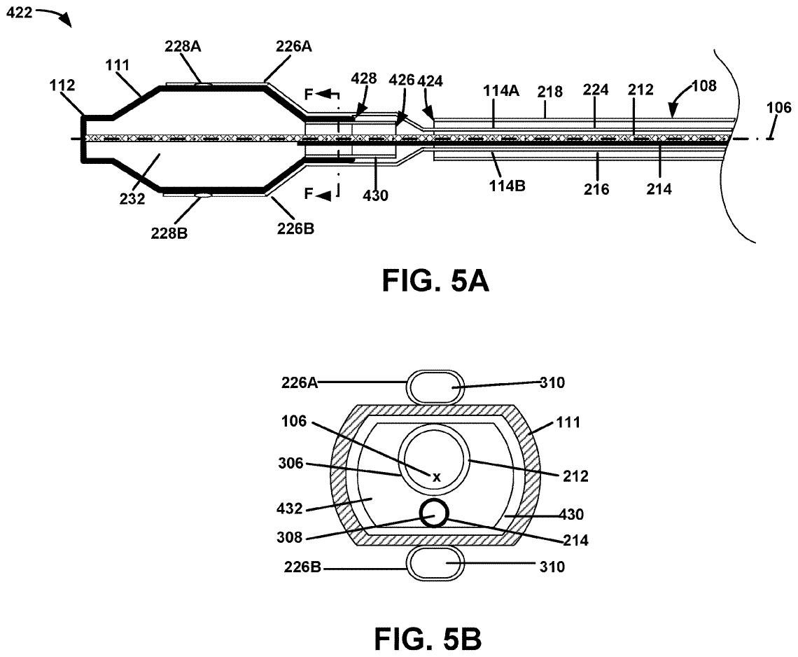

As discussed above an expandable member (e.g., expandable member 111, expandable member 404) may be configured to not block (e.g., occlude or otherwise obstruct) injection ports 228 disposed on the expandable member when the expandable member is in the collapsed configuration. FIGS. 6A-8B illustrate example expandable members in collapsed configurations such that one or more pleats of the expandable members do not occlude or obstruct injection ports 228 of catheter 102.

FIG. 6A is a conceptual diagram illustrating a schematic cross-section view of expandable member 111 of the catheter 102 of FIG. 2B, the cross-section being take along line E-E and in a direction orthogonal to longitudinal axis 206. Expandable member 111 is in a first collapsed configuration 500A in FIG. 6A. FIG. 6B is a conceptual diagram illustrating a view of the expandable member 111 of the catheter 102 of FIG. 6A in a second collapsed configuration 500B. As expandable member 111 collapses into first collapsed configuration 500A, e.g., due to removal of an inflation fluid from inner volume 232, expandable member 111 forms a plurality of major pleats 504A (also referred to as “protrusions 504A”), a plurality of minor pleats 504B (also referred to as “protrusions 504B”), and inner member 502.

Inner member 502 may be a part of expandable member 111 and may surround contain guidewire tube 212. The dimensions of inner member 502 may be defined, at least in part, by structure and/or properties of external portions 226 of injection tubes 114. Major pleats 504A and minor pleats 504B extend from inner member 502 and major pleats 504A define an outer diameter 506. In some examples, outer diameter 506 corresponds to a maximum outer diameter of expandable member 111 in the expanded configuration. In some examples, outer diameter 506 may be about 2 mm to about 10 mm. In some examples, as illustrated in FIG. 6A, minor pleats 504B extend a shorter distance (measured in a radial direction) away from inner member 502 than major pleats 504A. In other examples, major pleats 504A and minor pleats 504B may extend a same distance from inner member 502. Major pleats 504A and minor pleats 504B are positioned between external portions 226.

Once expandable member forms major pleats 504A and minor pleats 504B, major pleats 504A and minor pleats 504B (collectively referred to as “pleats 504”) may collapse radially inwards towards longitudinal axis 106 to form second collapsed configuration 500B. A clinician may navigate catheter 102 through vasculature or retract catheter 102 into an outer delivery sheath while expandable member 111 is in second collapsed configuration 500B. Pleats 504 may fold towards external portions 226, e.g., due to material properties of expandable member 111. Pleats 504 may fold over each other as pleats 504 collapse inwardly towards longitudinal axis 106. For example, as illustrated in FIG. 6B, major pleats 504A may fold at least partially over minor pleats 504B. When pleats 504 are in second collapsed configuration 500B, pleats 504 define outer diameter 508, wherein outer diameter 508 is a maximum outer diameter of catheter 102 when expandable member 111 is in a collapsed configuration (e.g., second collapsed configuration 500B) and is navigated within the vasculature of the patient. In some examples, outer diameter 508 may be between about 0.6 mm to about 3 mm.

When pleats 504 are folded in second collapsed configuration 500B, external portions 226 and injection ports 228 (not shown in FIG. 6B) are not blocked (e.g., occluded or otherwise obstructed by pleats 504). That is, expandable member 111 does not overlap injection ports 228 in the radial direction when expandable member 111 is in the first or second collapsed configurations 500A, 500B. In some examples, pleats 504 may fold around longitudinal axis 106 and around a portion of the perimeter of inner member 502 between adjacent external portions 226. Having external portions 226 and injection ports 228 not blocked by pleats 504 helps reduce an outer diameter 508 and reduces overall profile of catheter 102 to facilitate navigation of catheter 102 through vasculature. In addition, when a therapeutic agent is delivered via injection ports 228 while expandable member 111 is in second collapsed configuration 500B, e.g., as a part of testing of catheter 102 prior to use within the patient, the therapeutic agent may not release directly into expandable member 111, thereby minimizing adverse effects to expandable member 111 (e.g., to pleats 504) due to the delivery of the high pressure fluid through injection ports 228.

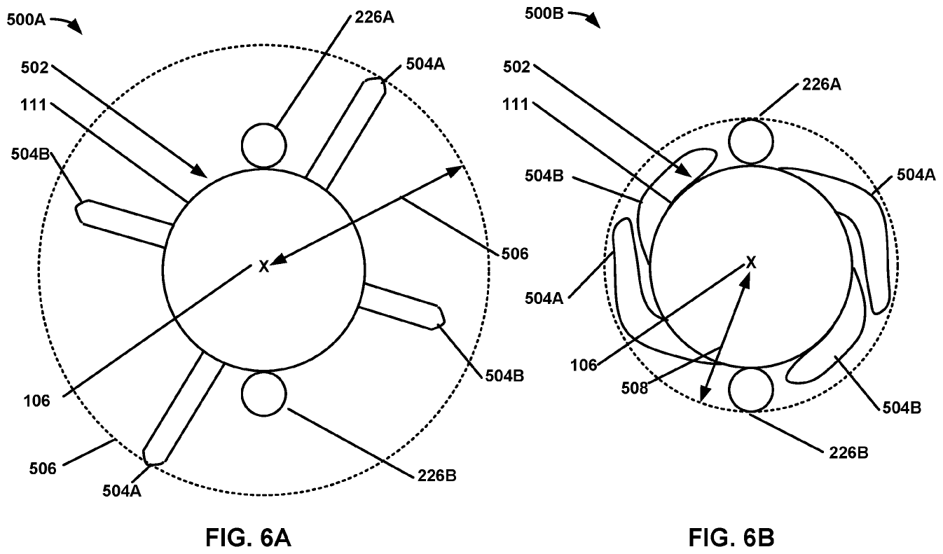

FIG. 7A is a conceptual diagram illustrating a schematic cross-section view of another example expandable member 512 of catheter 102 of FIG. 2B, the cross-section being taken along line F-F in a direction orthogonal to a longitudinal axis of the catheter, with expandable member 512 in a radially expanded configuration 510A. FIG. 7B is a conceptual diagram illustrating a cross-section view of the expandable member 512 of FIG. 6A in a collapsed configuration 510B. As illustrated in FIGS. 5C and 5D, expandable member 512 defines inner volume 514 and does not include guidewire tubes or any other inner members disposed within inner volume 514. Expandable member 512 also includes a plurality of channels 516 extending along longitudinal axis 106 along at least a portion of expandable member 512. External portions 226 of injection tubes 114 may be disposed within channels 516. Disposing injection tubes 114 within channels 516 may reduce outer diameter 506 of catheter 102 while expandable member 512 is in expanded configuration 510A.

When expandable member 512 transitions from expanded configuration 510A to collapsed configuration 510B, e.g., due to removal of an inflation fluid from inner volume 514, expandable member 512 forms pleats 518A, 518B (collectively referred to as “pleats 518”). Pleats 518 may collapse radially inwards towards longitudinal axis 106 and are positioned in a space between external portions 226, e.g., as illustrated in FIG. 7B. In the collapsed configuration 510B, pleats 518 and external portions 226 may define outer diameter 508. Pleats 518 may be positioned within the space between external portions 226 to reduce outer diameter 508 and/or prevent occlusion and/or obstruction of injection ports 228 on external portions 226. In some examples, as illustrated in FIG. 6B, expandable member 512 may form two pleats 518. In other examples, expandable member 512 may form three or more pleats 518. Pleats 518 may fold around longitudinal axis in a same direction (e.g., clockwise, as illustrated in FIG. 6B) or in different directions. Expandable member 512 may form pleats 518 and fold in a particular direction (e.g., clockwise or counter-clockwise) due to a material and/or structure of expandable member 512.

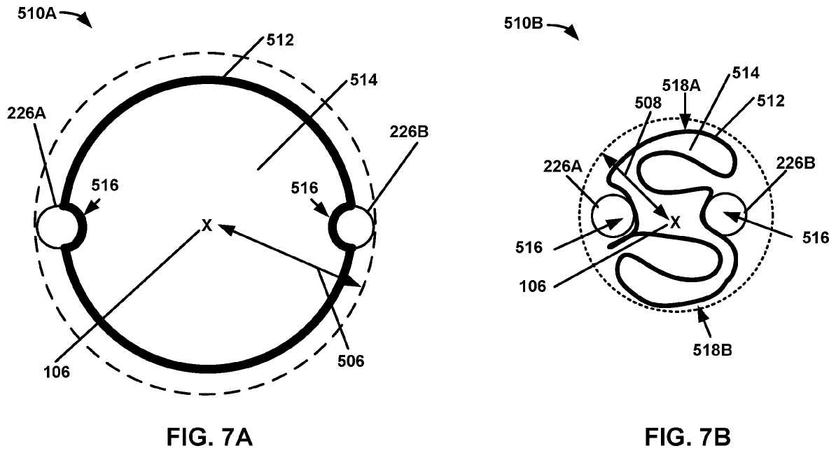

FIG. 8A is a conceptual diagram illustrating a schematic cross-section view of another example expandable member 111 of catheter 102 of FIG. 2B, the cross-section being taken along line F-F in a direction orthogonal to a longitudinal axis of the catheter, with expandable member 111 in a first collapsed configuration 500A. FIG. 8B is a conceptual diagram illustrating a view of the expandable member 111 of catheter 102 of FIG. 8A in a second collapsed configuration 500B.

As illustrated in FIGS. 8A and 8B, when expandable member 111 collapses into first collapsed configuration 500A, e.g., due to removal of an inflation fluid from inner volume 232, expandable member 111 forms minor pleats 520A and major pleats 520B (collectively “pleats 520”) around inner member 502. Adjacent pleats 520 may be interconnected and disposed between external portions 226 (e.g., along a perimeter of inner member 502 between adjacent external portions 226). Major pleats 520B may protrude a set length from inner member 502 and may define outer diameter 506. In some examples, as illustrated in FIG. 8A, major pleat 520B may be disposed between two minor pleats 520A. In some examples, expandable member 111 may form any number and/or arrangement of minor pleats 520A and/or major pleats 520B.

Once expandable member 111 forms pleats 520 in first collapsed configuration 500A, pleats 520 may fold radially inwards towards and around longitudinal axis 106, e.g., in the manner described in FIG. 6B. Pleats 520 may define outer diameter 508 and may fold around inner member 502 and between external portions 226 of injection tubes 114. A clinician may navigate catheter 102 through vasculature or retract catheter 102 into an outer delivery sheath while expandable member 111 is in second collapsed configuration 500B.

Pleats 520 may not block (e.g., occlude or otherwise obstruct) external portions 226 and/or injection ports 228, e.g., for the reasons described above.

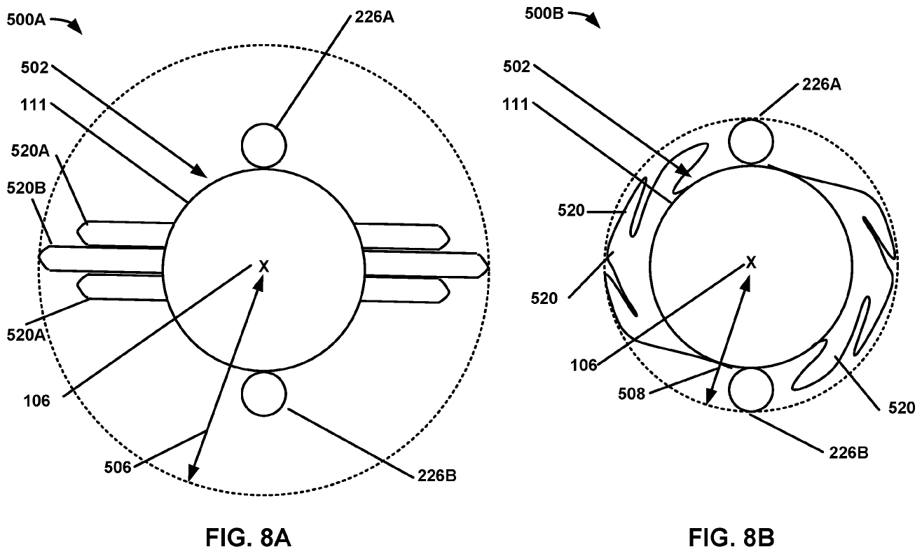

FIG. 9 is a flow diagram illustrating an example method of delivering therapy to a patient using a catheter (e.g., catheter 102) with multiple injection tubes 114. While FIG. 9 is primarily described with respect to therapy delivery to a target treatment site within a renal vessel of a patient, the example processes may be used for other target treatment sites and/or for other cavities within the patient. In some examples, catheter 102 may be disposed in the renal artery and may expel therapeutic agent via a plurality of injection tubes 114 disposed on expandable member 111. This may help reduce the need to manipulate catheter 102 at the target region and may increase efficacy of the therapy delivered to the patient.

In accordance with the technique of FIG. 9, a clinician inserts catheter 102 into vasculature of a patient (602). For example, the clinician may make an incision in the skin of the patient at an insertion site on the patient to reach a blood vessel of the patient. The clinician may then insert at least distal portion 108A of catheter 102 into the blood vessel. In some examples, prior to insertion of catheter 102 into the patient, the clinician may test the functionality of injection ports 228 on catheter 102, e.g., by expelling therapeutic agent from injection ports 228 while expandable member 111 is in a collapsed configuration. In the collapsed configuration, expandable member 111 may be folded around longitudinal axis 106 of catheter 102 to reduce an overall profile of catheter 102 during insertion and navigation of catheter 102. Expandable member 111 may be folded such that one or more pleats of expandable member 111 does not occlude or otherwise obstruct injection ports 228. The clinician may expel therapeutic agent from injection ports 228 without adverse effects to pleats of expandable member 111, e.g., due to occlusion or obstruction of injection ports 228 by the pleats.

The clinician may navigate catheter 102 to the target treatment site through the vasculature of the patient (604). The clinician may navigate catheter 102 using one or more imaging techniques such as X-ray imaging, fluoroscopy, or the like. In some examples, clinician advances catheter 102 over a guide member disposed within the vasculature (e.g., via an over-the-wire technique, rapid exchange technique, or the like). The clinician may advance catheter 102 within the vasculature until distal portion 108A reaches the target treatment site. In some examples, with regard to renal neuromodulation, the clinician may advance catheter 102 from a trans-radial entry point on the patient to a renal artery of the patient.

The clinician may transform expandable member 111 of catheter 102 into an expanded configuration (606). The clinician may insert an inflation fluid into inner volume 232 of expandable member 111. In some examples, the clinician inserts the inflation fluid into inner volume 232 via inner tube 214 (e.g., via a proximal end of inner tube 214 disposed within or near handle 104) and/or a catheter lumen of elongated body 108 (e.g., catheter lumen 216 or the like). The clinician may insert inflation fluid into expandable member 111 until expandable member 111 is in the expanded configuration. As expandable member 111 transitions to the expanded configuration, an outer surface of expandable member 111 may expand radially outwards of elongated body 108. At the expanded configuration, expandable member 111 may place injection ports 228 in apposition with wall tissue at the target treatment site. In some examples, expandable member 111 places injection ports 228 of injection tubes 114 radially around wall tissue such that the areas of influence of injection ports 228 substantially covers the target region and/or does not overlap.

In some examples, as described with reference to FIGS. 4A and 4B, after the clinician transforms expandable member 111 into the expanded configuration, the clinician may further expand injection ports 228 radially outwards by pulling back on an inner member (e.g., guidewire tube 212) connected to a distal end of expandable member 111. The pulling force may cause expandable member 111 to contract longitudinally and expand radially outwards away from longitudinal axis 106 and cause external portions 226 disposed on the outer surface of expandable member 111 to expand radially outwards towards wall tissue at the target region.

The clinician may deliver therapy to a target treatment site using one or more injection ports 228 on distal portion 108A of catheter 102 (608). The clinician may actuate catheter delivery system 100 to transmit a therapeutic agent at relatively high pressure (e.g., between about 5 MPa to about 18 MPa) through injection tube(s) 224. The pressurized therapeutic agent may exit injection ports 228 and penetrate the wall tissue in apposition with the injection ports 228. The clinician may continue to deliver the therapeutic agent to the target treatment site until the clinician determines that a user-determined amount of the therapeutic agent has been administered to the patient.

The clinician may transform expandable member 111 of catheter 102 into a collapsed configuration (610). The clinician may remove inflation fluid from inner volume 232 of expandable member 111. The clinician may remove the inflation fluid via inner tube 214 and/or the catheter lumen (e.g., catheter lumen 216, or the like). Once expandable member 111 is in the collapsed configuration, the clinician may remove catheter 102 from the vasculature and/or navigate distal portion 108A to a second target treatment site within the patient and deliver therapy to the second treatment site. In some examples expandable member 111, as a part of the transformation into the collapsed configuration, may form one or more pleats and fold around longitudinal axis and between external portions 226 of injection tubes 114, e.g., in accordance with the descriptions of any of FIGS. 6A-8B. When expandable member 111 is in the collapsed configuration, the overall profile of catheter 102 may be reduced to facilitate navigation of catheter 102 within the vasculature. In some examples, when expandable member 111 is in the collapsed configuration, expandable member 111 may not occlude or otherwise obstruct injection ports 228.

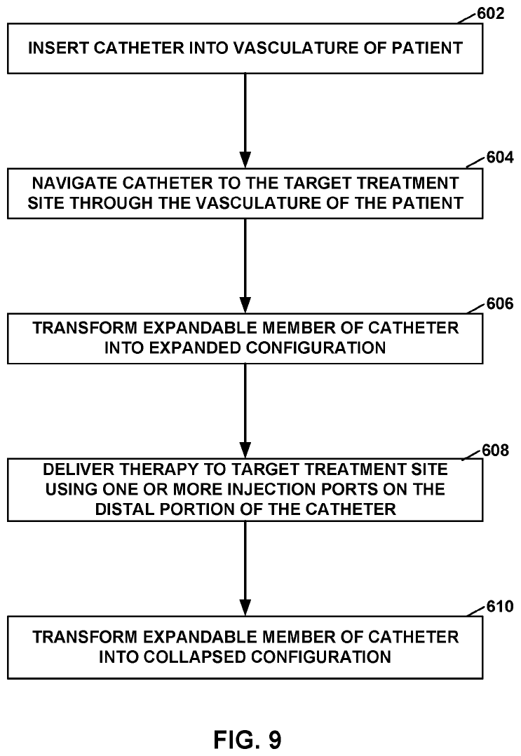

FIG. 10 is a conceptual illustration of an example process for accessing a renal artery and modulating renal nerves with the catheter of FIG. 1. While FIG. 10 illustrates the use of catheter 102 for renal neuromodulation, catheter 102 may be used for other therapies and treatments within another blood vessel or other hollow anatomical body within the human body. Catheter 102 is configured to delivery energy (e.g., radiofrequency energy, ultrasound energy, electrical stimulation energy, or the like) to one or more target treatment sites within a renal vessel. Catheter 102 provides access to the renal plexus (RP) through an intravascular path (P), such as a percutaneous access site in the femoral (illustrated), brachial, radial, or axillary artery to the target treatment sites within a respective renal artery (RA). By manipulating proximal portion 108B of elongated body 108 from outside the intravascular path (P), a clinician may advance at least distal portion 108A of elongated body 108 through the sometimes-tortuous intravascular path (P) and remotely manipulate distal portion 108A (FIG. 1) of elongated body 108. Distal portion 108A may be remotely manipulated by the clinician using the handle 104.

In the example illustrated in FIG. 10, distal portion 108A is delivered intravascularly to the treatment site using an inner member 136 in an over-the-wire (OTW) technique. Inner member 136 may be internal to catheter 102 (e.g., a guide wire, inner catheter, or the like) or external to catheter 102 (e.g., an outer sheath or the like). In some examples, inner member 136 is a navigation wire. Catheter 102 may define a passageway for receiving inner member 136 for delivery of catheter 102 using either an OTW or an RX technique. At the treatment site, inner member 136 can be at least partially withdrawn or removed relative to catheter 102 and distal portion 108A can transform into an expanded configuration (e.g., a helical configuration, a spiral configuration, or the like) for delivering ultrasound energy. In other examples, elongated body 108 may be self-steerable such that therapy delivery element 110 may be delivered to the target treatment site without the aid of inner member 136.

Renal modulation is the partial or complete incapacitation or other effective disruption of nerves of the kidneys (e.g., nerves terminating in the kidneys or in structures closely associated with the kidneys). In particular, renal neuromodulation can include inhibiting, reducing, and/or blocking neural communication along neural fibers (e.g., efferent and/or afferent neural fibers) of the kidneys. Such incapacitation can be long-term (e.g., permanent or for a period of months, years, or decades) or short-term (e.g., for periods of minutes, hours, days, or weeks). Renal neuromodulation is expected to contribute to the systemic reduction of sympathetic tone or drive and/or benefit at least some specific organs and/or other bodily structures innervated by sympathetic nerves. Accordingly, renal neuromodulation is expected to be useful in treating clinical conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end state renal disease, inappropriate fluid retention in heart failure, cardio-renal syndrome, polycystic kidney disease, polycystic ovary syndrome, osteoporosis, erectile dysfunction, and sudden death, among other conditions.

Renal neuromodulation can be electrically induced or induced in another suitable manner through the delivery of energy (RF energy, ultrasound energy, microwave energy, or the like). The target treatment site can be within or otherwise proximate to a renal lumen (e.g., a renal artery, a ureter, a renal pelvis, a major renal calyx, a minor renal calyx, or another suitable structure), and the target treatment site can include tissue at least proximate to a wall of the renal lumen. For example, with regard to a renal artery, a treatment procedure can include modulating nerves in the renal plexus, which lay intimately within or adjacent to the adventitia of the renal artery. The following discussion provides further details regarding patient anatomy and physiology as it may relate to renal denervation therapy. This section is intended to supplement and expand upon the previous discussion regarding the relevant anatomy and physiology, and to provide additional context regarding the disclosed technology and the therapeutic benefits associated with renal denervation. For example, several properties of the renal vasculature may inform the design of the target treatment devices and associate methods for achieving renal neuromodulation via intravascular access and impose specific design requirements for such devices. Specific design requirements may include accessing the renal artery, positioning distal portion 108A within the renal artery, delivering the therapy to targeted tissue, and/or effectively modulating the renal nerves with the therapy delivery device.

As noted previously, the sympathetic nervous system (SNS) is a branch of the autonomic nervous system along with the enteric nervous system and parasympathetic nervous system. It is always active at a basal level (called sympathetic tone) and becomes more active during times of stress. Like other parts of the nervous system, the sympathetic nervous system operated through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons join peripheral sympathetic neurons through synapses. Spinal cord sympathetic neurons are therefore called presynaptic (or preganglionic) neurons, while peripheral sympathetic neurons are called postsynaptic (or postganglionic neurons).

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation may elicit the release of adrenaline from the adrenal medulla.

Once released, norepinephrine and epinephrine bind adrenergic receptors on peripheral tissues. Binding to adrenergic receptors causes a neuronal and hormonal response. The physiologic manifestations include pupil dilation, increased heart rate, occasional vomiting, and increased blood pressure. Increased sweating is also seen due to binding of cholinergic receptors of the sweat glands.

The sympathetic nervous system is responsible for up-and down-regulating many homeostatic mechanisms in living organisms. Fibers from the SNS innervate tissues in almost every organ system, providing at least some regulatory function to physiological features as diverse as pupil diameter, gut motility, and urinary output. This response is also known as sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the sympathetic nervous system and indirectly via catecholamines secreted from the adrenal medulla.

Science typically looks at the SNS as an automatic regulation system, that is, one that operates without the intervention of conscious thought. Some evolutionary theorists suggest that the sympathetic nervous system operated in early organisms to maintain survival as the sympathetic nervous system is responsible for priming the body for action. One example of this priming is in the moments before waking, in which sympathetic outflow spontaneously increases in preparation for action.

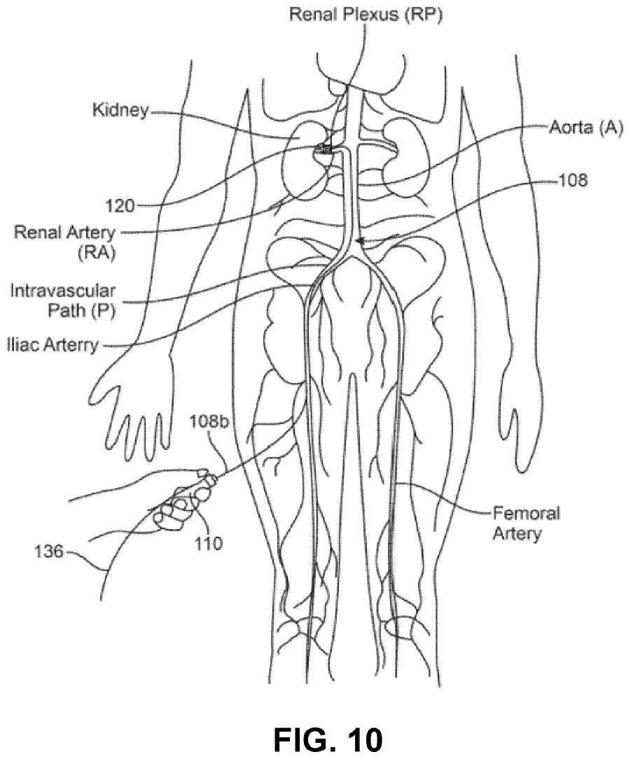

FIG. 11 is a conceptual illustration of an example sympathetic nervous system (SNS) illustrating how the brain communicates with the body via the SNS. As shown in FIG. 11, the SNS provides a network of nerves that allows the brain to communicate with the body. Sympathetic nerves originate inside the vertebral column, e.g., toward the middle of the spinal cord in the intermediolateral cell column (or lateral horn), beginning at the first thoracic segment of the spinal cord and are thought to extend to the second or third lumbar segments. Because SNS cells begin in the thoracic and lumbar regions of the spinal cord, the SNS is said to have a thoracolumbar outflow. Axons of sympathetic nerves leave the spinal cord through the anterior rootlet/root. The axons pass near the spinal (sensory) ganglion, where the axons enter the anterior rami of the spinal nerves. However, unlike somatic innervation, the axons separate out through white rami connectors which connect to either the paravertebral (which lie near the vertebral column) or prevertebral (which lie near the aortic bifurcation) ganglia extending alongside the spinal column.

To reach the target organs and glands, the axons should travel long distances in the body, and, to accomplish this, many axons relay their message to a second cell through synaptic transmission. The ends of the axons link across a space, the synapse, to the dendrites of the second cell. The first cell (the presynaptic cell) sends a neurotransmitter across the synaptic cleft where it activates the second cell (the postsynaptic cell). The message is then carried to the final destination.

In the SNS and other component of the peripheral nervous system, these synapses are made at sites called ganglia, discussed above. The cell that sends its fiber to the ganglion is called a preganglionic cell, while the cell whose fiber leaves the ganglion is called a postganglionic cell. As mentioned previously, the preganglionic cell of the SNS is located between the first thoracic (T1) segment and third lumbar (L3) segments of the spinal cord. Postganglionic cells have their cell bodies in the ganglia and send their axons to target organs or glands.

The ganglia include not just the sympathetic trunks but also the cervical ganglia (superior, middle, and inferior), which send sympathetic nerve fibers to the head and thorax organs, and the celiac and mesenteric ganglia, which send sympathetic fibers to the gut.

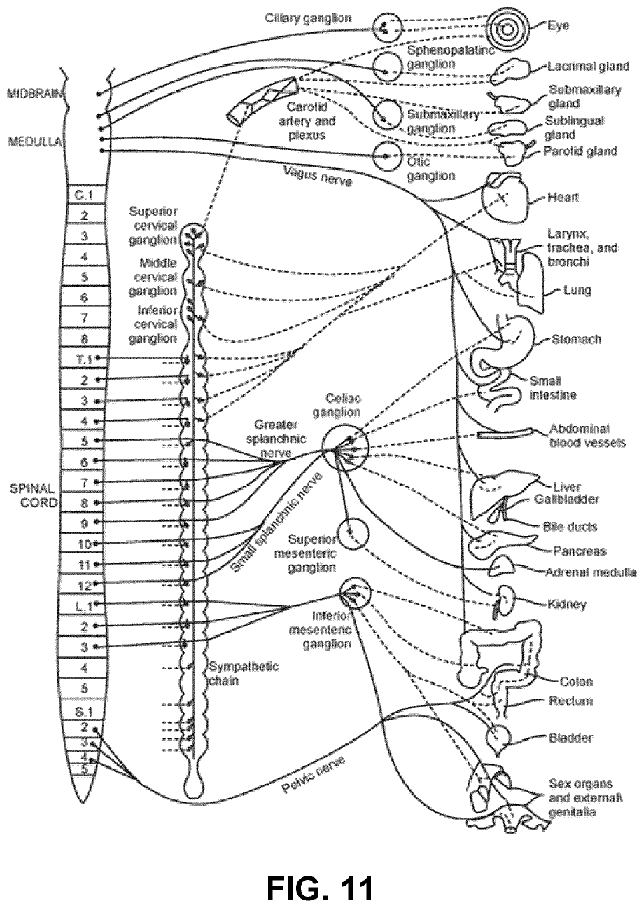

FIG. 12 is an enlarged anatomic view of nerves innervating a left kidney to form the renal plexus surrounding the left renal artery. As FIG. 12 shows, the kidney is innervated by the renal plexus (RP), which is intimately associated with the renal artery. The renal plexus (RP) is an autonomic plexus that surrounds the renal artery and is embedded within the adventitia of the renal artery. The renal plexus (RP) extends along the renal artery and is embedded within the adventitia of the renal artery. Fibers contributing to the renal plexus (RP) arise from the celiac ganglion, the superior mesenteric ganglion, the aorticorenal ganglion and the aortic plexus. The renal plexus (RP), also referred to as the renal nerve, is predominantly comprised of sympathetic components. There is no (or at least very minimal) parasympathetic innervation of the kidney.

Preganglionic neuronal cell bodies are located in the intermediolateral cell column of the spinal cord. Preganglionic axons pass through the paravertebral ganglia to become the lesser splanchnic nerve, the least splanchnic nerve, the first lumbar splanchnic nerve, the second lumbar splanchnic nerve, and travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus (RP) and are distributed to the renal vasculature.

Messages travel through the SNS in a bi-directional flow. Efferent messages may trigger changes in different parts of the body simultaneously. For example, the sympathetic nervous system may accelerate heart rate, widen bronchial passages, decrease motility (movement) of the large intestine, constrict blood vessels, increase peristalsis in the esophagus, cause pupil dilation, piloerection (goose bumps) and perspiration (sweating), or raise blood pressure. Afferent messages carry signals from various organs and sensory receptors in the body to other organs and, particularly, the brain.

Hypertension, heart failure, and chronic kidney disease are a few of the many disease states that result from chronic activation of the SNS, especially the renal sympathetic nervous system. Chronic activation of the SNS is a maladaptive response that drives the progression of theses disease states. Pharmaceutical management of the renin-angiotensin-aldosterone system (RAAS) has been a longstanding, but somewhat ineffective, approach for reducing over-activity of the SNS.

As mentioned above, the renal sympathetic nervous system has been identified as a major contributor to the complex pathophysiology of hypertension, states of volume overload (such as heart failure) and progressive renal disease, both experimentally and in humans. Studies employing radiotracer dilution methodology to measure overflow of norepinephrine from the kidneys to plasma revealed increased renal norepinephrine (NE) spillover rates in patients with essential hypertension, particularly so in young hypertensive subjects, which in concert with increased NE spillover from the heart, is consistent with the hemodynamic profile typically seen in early hypertension and characterized by an increased heart rate, cardiac output, and renovascular resistance. It is now known that essential hypertension is commonly neurogenic, often accompanied by pronounced sympathetic nervous system overactivity.

Activation of cardiorenal sympathetic nerve activity is even more pronounced in heart failure, as demonstrated by an exaggerated increase of NE overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration late, and left ventricular ejection fraction. These findings support the notion that treatment regimens that are designed to reduce renal sympathetic stimulation have the potential to improve survival in patients with heart failure.

Both chronic and end state renal disease in some patients are characterized by heightened sympathetic nervous activation. In patients with end state renal disease, plasma levels of norepinephrine above the media have been demonstrated to be predictive for both all-cause death and death from cardiovascular disease. This can also be true for patients suffering from diabetic or contrast nephropathy. There is compelling evidence suggesting that sensory afferent signals originating from the diseased kidneys are major contributors to initiating and sustaining elevated central sympathetic outflow in this patient group; this facilitates the occurrence of the well-known adverse consequences of chronic sympathetic over activity, such as hypertension, left ventricular hypertrophy, ventricular arrhythmias, sudden cardiac death, insulin resistance, diabetes, and metabolic syndrome.

Sympathetic nerves to the kidneys terminate in the blood vessels, the juxtaglomerular apparatus, and the renal tubules. Stimulation of the renal sympathetic nerves cause increased renin release, increased sodium (Na⁺) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation may be a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release), and diuretics (intended to counter the renal sympathetic mediated sodium and water retention). However, the current pharmacologic strategies can have significant limitations including limited efficacy, compliance issues, side effects, and others.

The kidneys communicate with integral structures in the central nervous system via renal sensory afferent nerves. Several forms of “renal injury” may induce activation of sensory afferent signals. For example, renal ischemia, reduction in stroke volume or renal blood flow, or an abundance of adenosine enzyme may trigger activation of afferent neural communication.

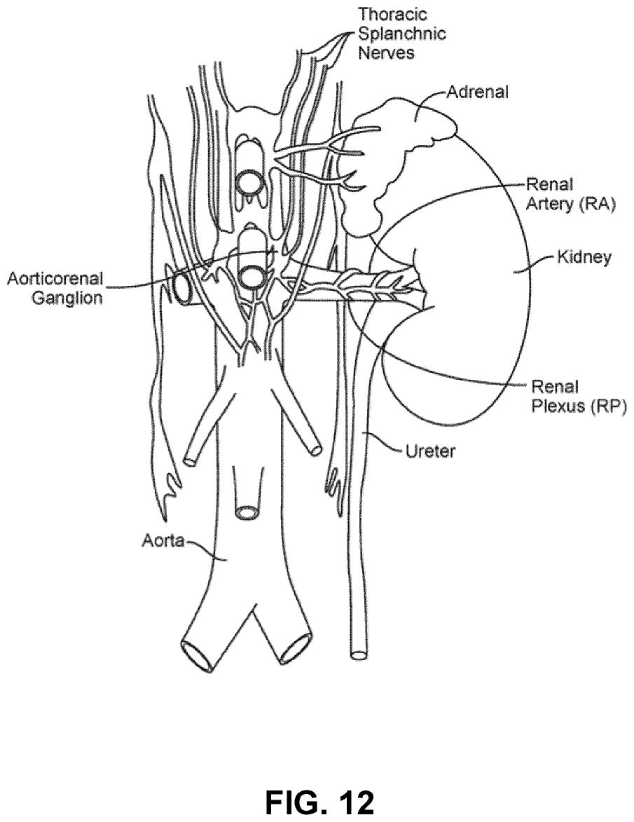

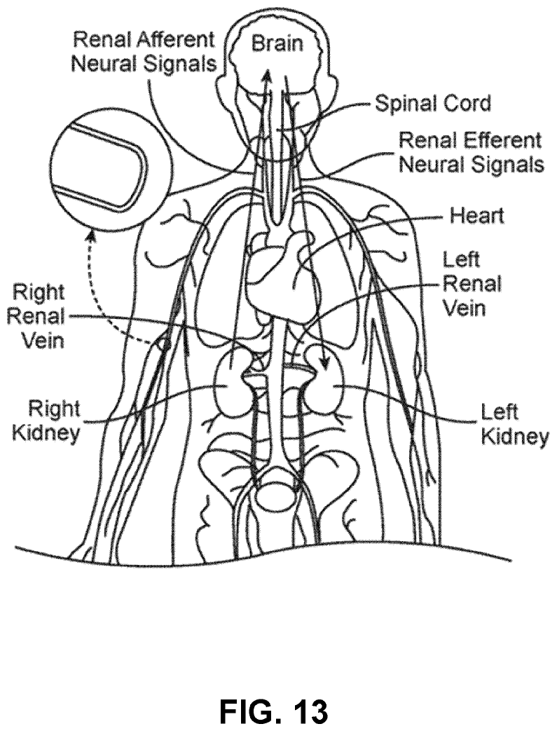

FIG. 13 is an anatomic view of a human body depicting neural efferent and afferent communication between the brain and kidneys. FIG. 14 is a conceptual view of a human body depicting neural efferent and afferent communication between the brain and kidneys. As shown in FIGS. 10 and 11, the afferent communication might be from kidney to the brain or might be from one kidney to the other kidney (via the central nervous system).

These afferent signals are centrally integrated and may result in increased sympathetic outflow. This sympathetic drive is directed towards the kidneys, thereby activating the RAAS and inducing increased renin secretion, sodium retention, volume retention, and vasoconstriction. Central sympathetic over activity also impacts other organs and bodily structures innervated by sympathetic nerves such as the heart and the peripheral vasculature, resulting in the described adverse effects of sympathetic activation, several aspects of which also contribute to the rise in blood pressure.

The physiology therefore suggests that (i) modulation of tissue with efferent sympathetic nerves will reduce inappropriate renin release, salt retention, and reduction of renal blood flow, and that (ii) modulation of tissue with afferent sensory nerves will reduce the systemic contribution to hypertension and other disease states associated with increased central sympathetic tone through its direct effect on the posterior hypothalamus as well as the contralateral kidney. In addition to the central hypotensive effects of afferent renal denervation, a desirable reduction of central sympathetic outflow to various other sympathetically innervated organs such as the heart and the vasculature is anticipated.

As provided above, renal denervation is likely to be valuable in the treatment of several clinical conditions characterized by increased overall and particularly renal sympathetic activity such as hypertension, metabolic syndrome, insulin resistance, diabetes, left ventricular hypertrophy, chronic end state renal disease, inappropriate fluid retention in heart failure, cardio-renal syndrome and sudden death. Since the reduction of afferent neural signals contributing to the systemic reduction of sympathetic tone/drive, renal denervation might also be useful in treating other conditions associate with systemic sympathetic hyperactivity. Accordingly, renal denervation may also benefit other organs and bodily structures innervated by sympathetic nerves, including those identified in FIG. 13. For example, as previously discussed, a reduction in central sympathetic drive may reduce the insulin resistance that afflicts people with metabolic syndrome and Type II diabetics. Additionally, patients with osteoporosis may also be sympathetically activated and might also benefit from the down regulation of sympathetic drive that accompanies renal denervation.

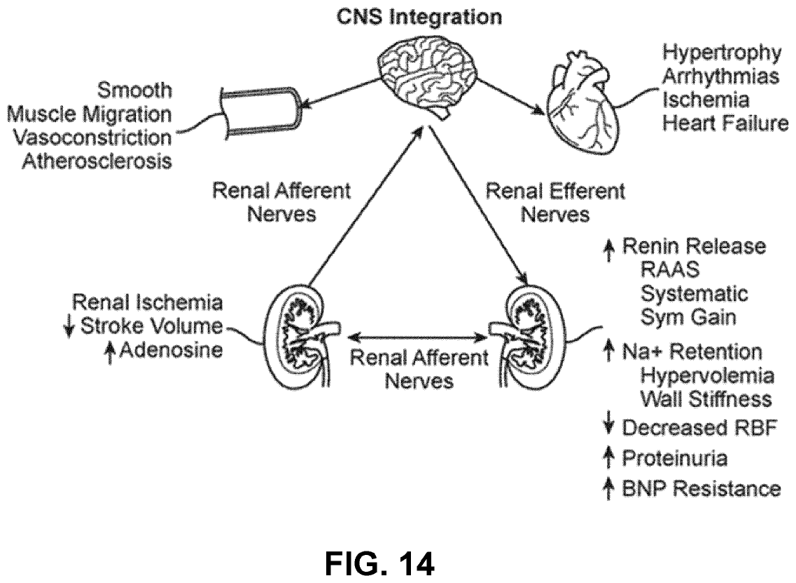

In accordance with the present technology neuromodulation of a left and/or right renal plexus (RP), which is intimately associated with a left and/or right renal artery, may be achieved through intravascular access. FIG. 15 is an anatomic view of the arterial vasculature of a human. As FIG. 15 shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries.

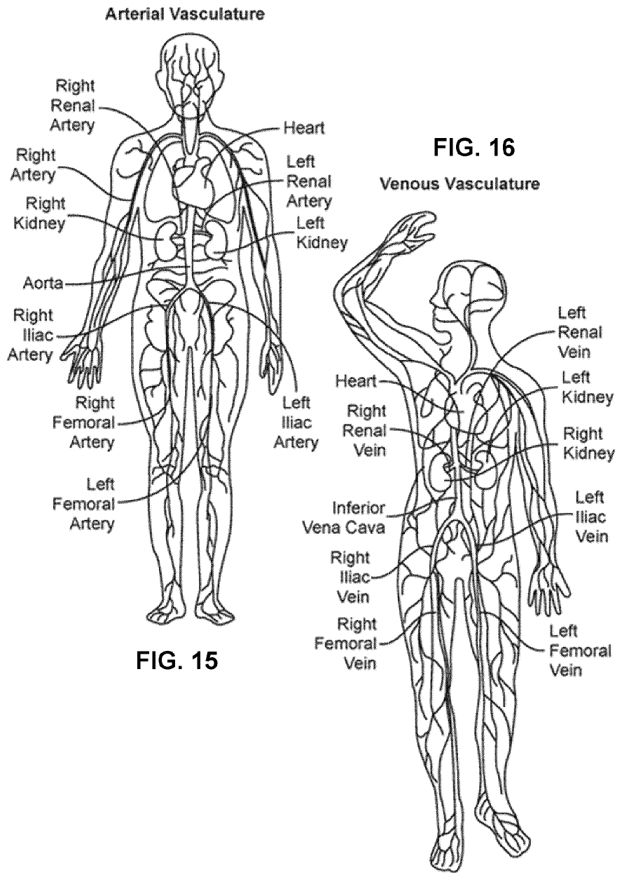

FIG. 16 is an anatomic view of the venous vasculature of a human. As FIG. 16 shows, the blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, the blood is pumped through the right ventricle into the lungs, where it is oxygenated. From the lungs, the oxygenated blood is conveyed into the left atrium. From the left atrium, the oxygenated blood is conveyed by the left ventricle back to the aorta.

The femoral artery may be accessed and cannulated at the base on the femoral triangle just inferior to the midpoint of the inguinal ligament. A catheter may be inserted percutaneously into the femoral artery through this access site, passed through the iliac artery and aorta, and placed into either the left or right renal artery. This comprises an intravascular path that offers minimally invasive access to a respective renal artery and/or other renal blood vessels.

The wrist, upper arm, and shoulder region provide other locations for introduction of catheters into the arterial system. For example, catheterization of either the radial, brachial, or axillary artery may be utilized in select cases. Catheters (e.g., catheter 102) introduced via these access points may be passed through the subclavian artery on the left side (or via the subclavian and brachiocephalic arteries on the right side), through the aortic arch, down the descending aorta and into the renal arteries using standard angiographic techniques. Other access sites can also be used to access the arterial system.

Since neuromodulation of a left and/or right renal plexus (RP) may be achieved in accordance with the present technology through intravascular access, properties and characteristics of the renal vasculature may impose constraints upon and/or inform the design of apparatus, systems, and methods for achieving such renal neuromodulation. Some of these properties and characteristics may vary across the patient population and/or within a specific patient across time, as well as in response to disease states, such as hypertension, chronic kidney disease, vascular disease, end-stage renal disease, insulin resistance, diabetes, metabolic syndrome, and the like. These properties and characteristics, as explained herein, may have bearing on the efficacy of the procedure and the specific design of the intravascular device. Properties of interest may include, for example, material/mechanical, spatial, fluid dynamic/hemodynamic and/or thermodynamic properties.

As discussed previously, a catheter may be advanced percutaneously into either the left or right renal artery via a minimally invasive intravascular path. However, minimally invasive renal arterial access may be challenging, for example, because as compared to some other arteries that are routinely accessed using catheters, the renal arteries are often extremely tortuous, may be of relatively small diameter, and/or may be of relatively short length. Furthermore, renal arterial atherosclerosis is common in many patients, particularly those with cardiovascular disease. Renal arterial anatomy also may vary significantly from patient to patient, which further complicates minimally invasive access. Significant inter-patient variation may be seen, for example, in relative tortuosity, diameter, length, and/or atherosclerotic plaque burden, as well as in the take-off angle at which a renal artery branches from the aorta. Further, some patients include multiple left renal arteries and/or right renal arteries. Apparatus, systems, and methods for achieving renal neuromodulation via intravascular access should account for these and other aspects of renal arterial anatomy and its variation across the patient population when minimally invasively accessing a renal artery.

In addition to complicating renal arterial access, specifics of the renal anatomy also complicate establishment of stable contact between neuromodulatory apparatus and a luminal surface or wall of a renal artery. For example, navigation can be impeded by the tight space within a renal artery, as well as tortuosity of the artery. Furthermore, establishing consistent contact is complicated by patient movement, respiration, and/or the cardiac cycle because these factors may cause significant movement of the renal artery relative to the aorta, and the cardiac cycle may transiently distend the renal artery (i.e., cause the wall of the artery to pulse).

The neuromodulatory apparatus may also be configured to allow for adjustable positioning and repositioning of distal portion 108A and therapy delivery elements 110 (FIG. 1) within the renal artery since location of treatment may also impact clinical efficacy. Additionally, variable positioning and repositioning of the neuromodulatory apparatus may prove to be useful in circumstances where the renal artery is particularly tortuous or where there are proximal branch vessels off the renal artery main vessel, making treatment in certain locations challenging.

As noted above, an apparatus positioned within a renal artery should be configured so that expandable distal portion 108A of catheter 102 may intimately contact the vessel wall and/or extend at least partially through the vessel wall. Renal artery vessel diameter, DRA, typically is in a range of about 2-10 mm, with most of the patient population having a DRA of about 4 mm to about 8 mm and an average of about 6 mm. Renal artery vessel length, LRA, between its ostium at the aorta/renal artery juncture and its distal branchings, generally is in a range of about 5-70 mm, and a significant portion of the patient population is in a range of about 20-50 mm. Since the target renal plexus is embedded within the adventitia of the renal artery, the composite Intima-Media Thickness, IMT, (i.e., the radial outward distance from the artery's luminal surface to the adventitia containing target neural structures) also is notable and generally is in a range of about 0.5-2.5 mm, with an average of about 1.5 mm. Although a certain depth of treatment is important to reach the target neural fibers, the treatment should not be too deep (e.g., >10 mm from inner wall of the artery) to avoid non-target tissue and anatomical structures such as anatomical structures of the digestive system of psoas muscle.

An additional property of the renal artery that may be of interest is the degree of renal motion relative to the aorta induced by respiration and/or blood flow pulsatility. A patient's kidney, which is located at the distal end of the renal artery, may move as much as 10 centimeters cranially with respiratory excursion. This may impart significant motion to the renal artery connecting the aorta and the kidney, thereby requiring from the neuromodulatory apparatus a unique balance of stiffness and flexibility to maintain contact between the energy delivery element and the vessel wall during cycles of respiration. Furthermore, the take-off angle between the renal artery and aorta may vary significantly between patients, and also may vary dynamically within a patient, e.g., due to kidney motion. The take-off angle generally may be in a range of about 30°-135°.

The above detailed descriptions of examples of the technology are not intended to be exhaustive or to limit the technology to the precise form disclosed above. Although specific examples of the technology are described above for illustrative purposes, various equivalent modifications are possible within the scope of the technology, as those skilled in the relevant art will recognize. For example, while steps are presented in a given order, alternative examples may perform steps in a different order. The various examples described herein may also be combined to provide further examples. All references cited herein are incorporated by reference as if fully set forth herein.

From the foregoing, it will be appreciated that specific examples of the present disclosure have been described herein for purposes of illustration, but that various modifications may be made without deviating from the present disclosure.

Certain aspects of the present disclosure described in the context of particular examples may be combined or eliminated in other examples. Further, while advantages associated with certain examples have been described in the context of those examples, other examples may also exhibit such advantages, and not all examples need necessarily exhibit such advantages to fall within the scope of the present disclosure. Accordingly, the present disclosure and associated technology can encompass other examples not expressly shown or described herein.

Further, although techniques have been described in which a neuromodulation catheter is positioned at a single location within a single renal artery, in other examples, the neuromodulation catheter may be repositioned to a second treatment site within a single renal artery (e.g., proximal or distal of the first treatment site, may be repositioned in a branch of the single artery, may be repositioned within a different renal vessel on the same side of the patient (e.g., a renal vessel associated with the same kidney of the patient), may be repositioned in a renal vessel on the other side of the patient (e.g., a renal vessel associated with the other kidney of the patient), or any combination thereof. At each location where the neuromodulation catheter is positioned, renal neuromodulation may be performed using any of the techniques described herein or any other suitable renal neuromodulation technique or any combination thereof.

Moreover, unless the word “or” is expressly limited to mean only a single term exclusive from the other items in reference to a list of two or more items, then the use of “or” in such a list is to be interpreted as including (a) any single item in list, (b) all of the items in the list, or (c) any combination of the items in the list. Additionally, the terms “about” or approximately,” when preceding a value, should be interpreted to mean plus or minus 10% of the value, unless otherwise indicated. Additionally, the term “comprising” is used throughout to mean including at least the recited feature(s) such that any greater number of the same feature and/or additional types of other features are not precluded.

Various aspects of the disclosure have been described, such as in the following examples. These and other aspects are within the scope of the claims.

Example 1: A catheter comprising: a catheter body; an expandable member connected to the catheter body, the expandable member being configured to expand from a collapsed configuration to an expanded configuration; and a plurality of injection tubes disposed on an outer surface of the expandable member, each of the plurality of injection tubes defining an injection tube lumen and an injection port in fluid communication with the respective injection tube lumen, wherein the expandable member is configured to not occlude the injection ports of the plurality of injection tubes when the expandable member is in the collapsed configuration.

Example 2: the catheter of example 1, wherein the expandable member is configured to place at least one of the injection ports of the plurality of injection tubes in apposition with a wall of a blood vessel of a patient when the expandable member is in the expanded configuration in the blood vessel.

Example 3: the catheter of example 1 or example 2, wherein the catheter is configured to deliver a therapy to a patient by at least delivering a therapeutic agent to tissue of the patient via the injection ports of the plurality of injection tubes.

Example 4: the catheter of any of examples 1-3, wherein the plurality of injection tubes comprises two injection tubes disposed 180 degrees apart.

Example 5: the catheter of any of examples 1-4, wherein the catheter body defines a catheter lumen, the catheter further comprising a fluid delivery tube disposed within the catheter lumen, wherein each injection tube of the plurality of injection tubes comprises a proximal portion disposed within the catheter lumen and in fluid communication with the fluid delivery tube.

Example 6: the catheter of example 5, wherein each injection tube of the plurality of injection tubes exits the catheter lumen and extends along an outer surface of the catheter body proximal to a proximal end of the expandable member.

Example 7: the catheter of any of examples 1-6, wherein when the expandable member transforms from the expanded configuration to the collapsed configuration, the expandable member is configured to fold radially inwards and around a longitudinal axis of the catheter, wherein when folded radially inwards, the expandable member is configured to not occlude the injection ports of the plurality of injection tubes.

Example 8: the catheter of example 7, wherein when the expandable member is in the collapsed configuration, the expandable member is configured to form one or more pleats extending radially outwards of the longitudinal axis.

Example 9: the catheter of example 8, wherein the one or more pleats is configured to fold towards the longitudinal axis and into a region between two injection tubes of the plurality of injection tubes.

Example 10: the catheter of example 7, wherein the expandable member is configured to form two or more pleats between the plurality of injection tubes while the expandable member is in the collapsed configuration.

Example 11: the catheter of any of examples 1-10, wherein the catheter body defines a catheter lumen, the catheter further comprising a guide member disposed within the catheter lumen.

Example 12: the catheter of example 11, wherein the guide member comprises a guidewire tube defining a guidewire lumen configured to receive a guidewire.

Example 13: the catheter of example 11, wherein the guide member comprises one or more pull wires disposed within the catheter lumen.

Example 14: the catheter of any of examples 1-13, wherein the catheter body defines a catheter lumen, the catheter further comprising an inner tube disposed within the catheter lumen, the inner tube defining an inner lumen in fluid communication with the expandable member.

Example 15: the catheter of any of examples 1-14, wherein the expandable member comprises a compliant balloon.

Example 16: the catheter of any of examples 1-15, wherein the expandable member defines a plurality of channels when the expandable member is in an expanded configuration, and wherein each injection tube of the plurality of injection tubes is disposed within a channel of the plurality of channels.

Example 17: a catheter comprising: a catheter body defining a catheter lumen; an expandable member connected to the catheter body; an inner member disposed within the catheter lumen; and a plurality of injection tubes disposed on an outer surface of the expandable member, each of the plurality of injection tubes defining an injection tube lumen and an injection port in fluid communication with the respective injection tube lumen, wherein in response to proximal movement of the inner member relative to the expandable member while the expandable member is in an expanded configuration, the expandable member is configured to expand radially outwards.

Example 18: the catheter of example 17, wherein when the expandable member is expanded radially outwards in a blood vessel of a patient, the expandable member is configured to push the plurality of injection tubes away from a longitudinal axis of the catheter and place at least one of the injection ports of the plurality of injection tubes in apposition with a wall of the blood vessel.

Example 19: the catheter of any of examples 17 and 18, wherein the catheter is configured to deliver a therapy to a patient by at least delivering a therapeutic agent to tissue of the patient via the injection ports of the plurality of injection tubes.

Example 20: the catheter of any of examples 17-19, wherein the expandable member is configured to not occlude the injection ports of the plurality of injection tubes when the expandable member is in a collapsed configuration.

Example 21: the catheter of any of examples 17-20, wherein the plurality of injections tubes comprises two injection tubes disposed 180 degrees apart.

Example 22: the catheter of any of examples 17-21, further comprising a fluid delivery tube disposed within the catheter lumen, wherein each injection tube of the plurality of injection tubes comprises a proximal portion disposed within the catheter lumen and in fluid communication with the fluid delivery tube.

Example 23: the catheter of example 22, wherein each injection tube of the plurality of injection tubes exits the catheter lumen and extends along an outer surface of the catheter body at a point proximal to a proximal end of the expandable member.

Example 24: the catheter of any of examples 17-23, wherein when the expandable member transforms from the expanded configuration to a collapsed configuration, the expandable member is configured to fold radially inwards and around a longitudinal axis of the catheter.

Example 25: the catheter of example 24, wherein when the expandable member is in the collapsed configuration, the expandable member is configured to form one or more pleats extending radially outwards of the longitudinal axis.

Example 26: the catheter of example 25, wherein the one or more pleats is configured to fold towards the longitudinal axis and into a region between the inner member and the plurality of injection tubes.

Example 27: the catheter of any of examples 17-26, wherein the inner member is configured to deflect a distal end of the catheter.

Example 28: the catheter of example 27, wherein the inner member comprises a guidewire tube defining a guidewire lumen.

Example 29: the catheter of example 28, further comprising a guide member disposed within the guidewire lumen.

Example 30: the catheter of example 29, wherein the guide member comprises a guidewire.

Example 31: the catheter of any of examples 17-30, further comprising an inner tube disposed within the catheter lumen, the inner tube defining an inner lumen in fluid communication with the expandable member.

Example 32: the catheter of any of examples 17-31, wherein the expandable member comprises a compliant balloon.

Example 33: a method of delivering therapy to tissue of a patient, the method comprising: navigating a catheter through vasculature of the patient to a target treatment site, wherein the catheter comprises: a catheter body; an expandable member connected to the catheter body; and a plurality of injection tubes disposed on an outer surface of the expandable member, each of the plurality of injection tubes defining an injection tube lumen and an injection port in fluid communication with the respective injection tube lumen; expanding the expandable member of the catheter from a collapsed configuration to an expanded configuration; and delivering therapy to the target treatment site via the injection ports of the plurality of injection tubes, wherein the expandable member does not occlude the injection ports when the expandable member is in the collapsed configuration.

Example 34: the method of example 33, wherein expanding the expandable member of the catheter to the expanded configuration comprises placing at least one of the injection ports of the plurality of injection tubes in apposition with wall of a blood vessel of the patient at the target treatment site.

Example 35: the method of any of examples 33 and 34, wherein delivering the therapy to the target treatment site comprises delivering a therapeutic agent to the tissue of the patient via the injection ports of the plurality of injection tubes.

Example 36: the method of any of examples 33-35, wherein the plurality of injection tubes comprises two injection tubes disposed 180 degrees apart.

Example 37: the method of any of examples 33-36, wherein the catheter body defines a catheter lumen, wherein the catheter further comprises a fluid delivery tube disposed within the catheter lumen, wherein each injection tube of the plurality of injection tubes comprises a proximal portion disposed within the catheter lumen and in fluid communication with the fluid delivery tube.

Example 38: the method of example 37, wherein each injection tube of the plurality of injection tubes exits the catheter lumen and extends along an outer surface of the catheter body proximal to a proximal end of the expandable member.

Example 39: the method of any of examples 33-38, further comprising transforming the expandable member from the expanded configuration to the collapsed configuration, wherein transforming the expandable member from the expanded configuration to the collapsed configuration comprises folding the expandable member radially inwards and around a longitudinal axis of the catheter.

Example 40: the method of example 39, wherein folding the expandable member radially inwards comprises: forming one or more pleats in the expandable member; and folding the one or more pleats radially inwards and around the longitudinal axis of the catheter.

Example 41: the method of example 40, wherein transforming the expandable member from the expanded configuration to the collapsed configuration further comprises folding the one or more pleats into a region between two injection tubes of the plurality of injection tubes.

Example 42: the method of example 41, wherein the expandable member is configured to form two or more pleats between the plurality of injection tubes while the expandable member is in the collapsed configuration.

Example 43: the method of any of examples 33-42, wherein the catheter body defines a catheter lumen, and wherein the catheter further comprises a guide member disposed within the catheter lumen.

Example 44: the method of example 43, wherein the guide member comprises a guidewire tube defining a guidewire lumen configured to receive a guidewire, and wherein navigating the catheter through vasculature of the patient to the target treatment site comprises navigating the catheter through vasculature of the patient over the guidewire.

Example 45: the method of any of examples 33-44, wherein the expandable member comprises a compliant balloon.

Example 46: the method of any of examples 33-45, wherein the expandable member defines a plurality of channels when the expandable member is in an expanded configuration, and wherein each injection tube of the plurality of injection tubes is disposed within a channel of the plurality of channels.

Example 47: the method of any of examples 33-46, wherein expanding the expandable member comprises delivering a fluid into an inner volume of the expandable member.

Example 48: the method of any of examples 33-47, where the target treatment site comprises a first target treatment site, the method further comprising: transforming the expandable member from the expanded configuration to the collapsed configuration; navigating the catheter through the vasculature of the patient to a second target treatment site; transforming the expandable member from the collapsed configuration to the expanded configuration; and delivering the therapy to the second target treatment site via the injection ports of the plurality of injection tubes.

Example 49: a method of delivering therapy to tissue of a patient, the method comprising: navigating a catheter through vasculature of the patient to a target treatment site, wherein the catheter comprises: a catheter body defining a catheter lumen; an expandable member connected to the catheter body; an inner member disposed within the catheter lumen; and a plurality of injection tubes disposed on an outer surface of the expandable member, each of the plurality of injection tubes defining an injection tube lumen and an injection port in fluid communication with the respective injection tube lumen; expanding the expandable member of the catheter from a collapsed configuration to an expanded configuration; retracting the inner member proximally relative to the catheter body, the retraction of the inner member causing the expandable member to expand further radially outwards from the expanded configuration and towards the tissue of the target treatment site; and delivering therapy to the target treatment site via the injection ports of the plurality of injection tubes.

Example 50: the method of example 49, wherein retracting the inner member proximally expands the plurality of injection tubes radially outwards and place at least one injection port of the plurality of injection tubes in apposition with a wall of a blood vessel at the target treatment site.

Example 51: the method of any of examples 49 and 50, wherein delivering the therapy to the target treatment site comprises delivering a therapeutic agent to tissue of the patient at the target treatment site via the injection ports of the plurality of injection tubes.

Example 52: the method of any of examples 49-51, wherein the expandable member is configured to not occlude the injection ports of the plurality of injection tubes when the expandable member is in the collapsed configuration.

Example 53: the method of any of examples 49-52, wherein the plurality of injection tubes comprises two injection tubes disposed 180 degrees apart.

Example 54: the method of any of examples 49-53, wherein the catheter further comprises a fluid delivery tube disposed within the catheter lumen, wherein each injection tube of the plurality of injection tubes comprises a proximal portion disposed within the catheter lumen and in fluid communication with the fluid delivery tube.

Example 55: the method of any of examples 49-54, wherein each injection tube of the plurality of injection tubes exit the catheter lumen and extends along an outer surface of the catheter body at a point proximal to a proximal end of the expandable member.

Example 56: the method of any of examples 49-55, wherein the transforming the expandable member to the collapsed configuration comprises folding the expandable member radially inwards and around a longitudinal axis of the catheter.

Example 57: the method of example 56, wherein folding the expandable member comprises: forming one or more pleats in the expandable member; and folding the one or more pleats radially inwards and around the longitudinal axis.

Example 58: the method of example 57, wherein folding the one or more pleats radially inwards and around the longitudinal axis comprises folding the one or more pleats towards the longitudinal axis and into a region between the inner member and the plurality of injection tubes.

Example 59: the method of any of examples 49-58, wherein navigating the catheter through the vasculature of the patient to the target treatment site comprising deflecting a distal end of the catheter using the inner member.

Example 60: the method of any of examples 49-59, wherein the expandable member comprises a compliant balloon.

Further disclosed herein is the subject-matter of the following clauses:
1. A catheter comprising:
   a catheter body;
   an expandable member connected to the catheter body, the expandable member being configured to expand from a collapsed configuration to an expanded configuration; and
   a plurality of injection tubes disposed on an outer surface of the expandable member, each of the plurality of injection tubes defining an injection tube lumen and an injection port in fluid communication with the respective injection tube lumen,
   wherein the expandable member is configured to not occlude the injection ports of the plurality of injection tubes when the expandable member is in the collapsed configuration.
2. The catheter of clause 1, wherein the expandable member is configured to place at least one of the injection ports of the plurality of injection tubes in apposition with a wall of a blood vessel of a patient when the expandable member is in the expanded configuration in the blood vessel.
3. The catheter of clause 1 or 2, wherein the catheter is configured to deliver a therapy to a patient by at least delivering a therapeutic agent to tissue of the patient via the injection ports of the plurality of injection tubes.
4. The catheter of any of clauses 1-3, wherein the plurality of injection tubes comprises two injection tubes disposed 180 degrees apart.
5. The catheter of any of clauses 1-4, wherein the catheter body defines a catheter lumen, the catheter further comprising a fluid delivery tube disposed within the catheter lumen, wherein each injection tube of the plurality of injection tubes comprises a proximal portion disposed within the catheter lumen and in fluid communication with the fluid delivery tube.
6. The catheter of clause 5, wherein each injection tube of the plurality of injection tubes exits the catheter lumen and extends along an outer surface of the catheter body proximal to a proximal end of the expandable member.
7. The catheter of any of clauses 1-6, wherein when the expandable member transforms from the expanded configuration to the collapsed configuration, the expandable member is configured to fold radially inwards and around a longitudinal axis of the catheter, wherein when folded radially inwards, the expandable member is configured to not occlude the injection ports of the plurality of injection tubes.
8. The catheter of clause 7, wherein when the expandable member is in the collapsed configuration, the expandable member is configured to form one or more pleats extending radially outwards of the longitudinal axis, and wherein the one or more pleats is configured to fold towards the longitudinal axis and into a region between two adjacent injection tubes of the plurality of injection tubes.
9. The catheter of clause 7, wherein the expandable member is configured to form two or more pleats between the plurality of injection tubes while the expandable member is in the collapsed configuration.
10. The catheter of any of clauses 1-9, wherein the expandable member comprises a compliant balloon.
11. The catheter of any of clauses 1-10, wherein the expandable member defines a plurality of channels when the expandable member is in an expanded configuration, and wherein each injection tube of the plurality of injection tubes is disposed within a channel of the plurality of channels.
12. The catheter of any one of clauses 1-11, further comprising:
   an inner member disposed within a catheter lumen of the catheter body, wherein, in response to proximal movement of the inner member relative to the expandable member while the expandable member is in an expanded configuration, the expandable member is configured to expand radially outwards.
13. The catheter of clause 12, wherein the inner member is configured to deflect a distal end of the catheter.
14. The catheter of clause 12 or 13, wherein the inner member comprises a guidewire tube defining a guidewire lumen, further comprising a guide member disposed within the guidewire lumen.
15. The catheter of clause 14, wherein the guide member comprises a guidewire.

1. A catheter comprising:
a catheter body;
an expandable member connected to the catheter body, the expandable member being configured to expand from a collapsed configuration to an expanded configuration; and
a plurality of injection tubes disposed on an outer surface of the expandable member, each of the plurality of injection tubes defining an injection tube lumen and an injection port in fluid communication with the respective injection tube lumen,
wherein the expandable member is configured to not occlude the injection ports of the plurality of injection tubes when the expandable member is in the collapsed configuration.

2. The catheter of claim 1, wherein the expandable member is configured to place at least one of the injection ports of the plurality of injection tubes in apposition with a wall of a blood vessel of a patient when the expandable member is in the expanded configuration in the blood vessel.

3. The catheter of claim 1, wherein the catheter is configured to deliver a therapy to a patient by at least delivering a therapeutic agent to tissue of the patient via the injection ports of the plurality of injection tubes.

4. The catheter of claim 1, wherein the plurality of injection tubes comprises two injection tubes disposed 180 degrees apart.

5. The catheter of claim 1, wherein the catheter body defines a catheter lumen, the catheter further comprising a fluid delivery tube disposed within the catheter lumen, wherein each injection tube of the plurality of injection tubes comprises a proximal portion disposed within the catheter lumen and in fluid communication with the fluid delivery tube.

6. The catheter of claim 5, wherein each injection tube of the plurality of injection tubes exits the catheter lumen and extends along an outer surface of the catheter body proximal to a proximal end of the expandable member.

7. The catheter of claim 1, wherein when the expandable member transforms from the expanded configuration to the collapsed configuration, the expandable member is configured to fold radially inwards and around a longitudinal axis of the catheter, wherein when folded radially inwards, the expandable member is configured to not occlude the injection ports of the plurality of injection tubes.

8. The catheter of claim 7, wherein when the expandable member is in the collapsed configuration, the expandable member is configured to form one or more pleats extending radially outwards of the longitudinal axis, and wherein the one or more pleats is configured to fold towards the longitudinal axis and into a region between two adjacent injection tubes of the plurality of injection tubes.

9. The catheter of claim 7, wherein the expandable member is configured to form two or more pleats between the plurality of injection tubes while the expandable member is in the collapsed configuration.

10-11. (canceled)

12. The catheter of claim 1, further comprising:
an inner member disposed within a catheter lumen of the catheter body, wherein, in response to proximal movement of the inner member relative to the expandable member while the expandable member is in an expanded configuration, the expandable member is configured to expand radially outwards.

13-15. (canceled)

16. A catheter comprising:
a catheter body defining a catheter lumen;
an expandable member connected to the catheter body;
an inner member disposed within the catheter lumen; and
a plurality of injection tubes disposed on an outer surface of the expandable member, each of the plurality of injection tubes defining an injection tube lumen and an injection port in fluid communication with the respective injection tube lumen,
wherein in response to proximal movement of the inner member relative to the expandable member while the expandable member is in an expanded configuration, the expandable member is configured to expand radially outwards.

17. The catheter of claim 16, wherein when the expandable member is expanded radially outwards in a blood vessel of a patient, the expandable member is configured to push the plurality of injection tubes away from a longitudinal axis of the catheter and place at least one of the injection ports of the plurality of injection tubes in apposition with a wall of the blood vessel.

18. The catheter of claim 16, wherein the expandable member is configured to not occlude the injection ports of the plurality of injection tubes when the expandable member is in a collapsed configuration.

19. The catheter of claim 16, further comprising a fluid delivery tube disposed within the catheter lumen, wherein each injection tube of the plurality of injection tubes comprises a proximal portion disposed within the catheter lumen and in fluid communication with the fluid delivery tube.

20. The catheter of claim 19, wherein each injection tube of the plurality of injection tubes exits the catheter lumen and extends along an outer surface of the catheter body at a point proximal to a proximal end of the expandable member.

21. The catheter of claim 16, wherein when the expandable member transforms from the expanded configuration to a collapsed configuration, the expandable member is configured to fold radially inwards and around a longitudinal axis of the catheter.

22. The catheter of claim 21, wherein when the expandable member is in the collapsed configuration, the expandable member is configured to form one or more pleats extending radially outwards of the longitudinal axis.

23. The catheter of claim 22, wherein the one or more pleats is configured to fold towards the longitudinal axis and into a region between the inner member and the plurality of injection tubes

24. A method of delivering therapy to tissue of a patient, the method comprising:
navigating a catheter through vasculature of the patient to a target treatment site, wherein the catheter comprises:
a catheter body;
an expandable member connected to the catheter body; and
a plurality of injection tubes disposed on an outer surface of the expandable member, each of the plurality of injection tubes defining an injection tube lumen and an injection port in fluid communication with the respective injection tube lumen;
expanding the expandable member of the catheter from a collapsed configuration to an expanded configuration; and
causing delivery of therapy to the target treatment site via the injection ports of the plurality of injection tubes,
wherein the expandable member does not occlude the injection ports when the expandable member is in the collapsed configuration.

25. The method of claim 24, wherein expanding the expandable member of the catheter to the expanded configuration comprises placing at least one of the injection ports of the plurality of injection tubes in apposition with wall of a blood vessel of the patient at the target treatment site.
